(19) United States
(12) Patent Application Publication
(10) Pub. No.: US 2019/0232968 A1
(43) Pub. Date: Aug. 1, 2019

(54) VEHICLE ADJUSTMENT

(71) Applicant: McLaren Applied Technologies Limited, Surrey (GB)

(72) Inventors: William Oliver James Reynolds, Surrey (GB); Robert Bowyer, London (GB); Andrew Murray Charles Odhams, Surrey (GB); Duncan James Bradley, Surrey (GB); Caleb Allan Sawade, Surrey (GB)

(21) Appl. No.: 16/339,603

(22) PCT Filed: Oct. 4, 2017
(86) PCT No.: PCT/GB2017/052967

§ 371 Date: Apr. 4, 2019

(30) Foreign Application Priority Data
Oct. 4, 2016 (GB) 1616865.0

(51) Int. Cl. B60W 50/00; B62J 99/00; A61G 5/10; B62B 9/00; A63C 17/01; B62K 11/00; B62K 3/00; A61B 5/00; A61B 5/0205; A61B 5/01; A61B 5/145; A61B 5/107

(52) U.S. Cl. CPC B60W 50/0098; A61B 5/021; A61G 5/10; B62B 9/00; A63C 17/01; B62K 11/00; B62K 3/00; A61B 5/6895; A61B 5/6894; A61B 5/6896; A61B 5/02055; A61B 5/01; A61B 5/14532; A61B 5/4519; A61B 5/1071; B62J 2099/0013; B60W 2550/10; B60W 2550/142; B60W 2550/148; B60W 2550/12; B62J 2099/002; B62K 2207/02; B62K 2207/04; B60W 2530/00; B60W 2530/20; B60W 2510/1005; B60W 2520/18; B60W 2520/16; B60W 2520/10; B60W 2520/105; B60W 2540/04; B60W 2540/22; B62J 99/00

(57) ABSTRACT

Disclosed is a vehicle comprising a frame component having an adjustable physical property; and a controller configured to receive data relating to the vehicle in use and, based on received data, determine a value of the physical property of the frame component. A method, a computer program and a control system are also disclosed.

### FIELD

This invention relates to a vehicle having one or more frame components which have an adjustable physical property, such as a physical property whose value affects a performance metric of the vehicle. The invention also relates to a control system, a method and a computer program product.

### BACKGROUND

Most vehicles are not designed and built for a specific user or terrain on which the vehicle is to be used, but rather, tend to be designed with an average user or, in some cases, a generic type of user or terrain in mind. For example, most people would buy a bicycle “off-the-shelf”. Different makes and models of bicycles do vary in their characteristics, for example frame size and mass, and generally have a limited number of adjustable features. For example, it is usually possible to manually adjust saddle height, and the saddle post is provided with a sufficient length to cater for a variety of user-preferred heights.

The limited adjustability of a small number of components of a vehicle such as a bicycle means that the vehicle tends not to be optimized for the user or the conditions. Some attempts have been made to allow some components to be adjustable for the user or conditions to a limited extent. For example, hydraulically-adjustable saddle posts are available. These can allow a pre-load adjustment to take account of the weight of the rider. They can also allow adjustment of seat post length, and hence saddle height relative to frame, during use by means of a user-operable lever that in turn operates the hydraulics. Thus the rider can judge the upcoming conditions and decide accordingly on a saddle height and then operate the lever until the desired height is reached. For example, it may be advantageous to lower the saddle prior to a difficult descent.

Whilst a saddle adjustment system of the type described above may improve handling of the bicycle and hence the user's experience, its scope is limited. Firstly, handling, comfort and safety may depend on more parameters of the bicycle than saddle height. For example, user comfort and ability to handle the bicycle might be affected by a distance between the saddle and handlebars, front or rear triangle layouts, the chainstay length or the angle at which they prefer to sit on the bicycle. This in turn may depend not only on user mass, but also on the user's dimensions such as length of arms, legs and body or the user's mass distribution. Another factor is user strength, which in turn affects forces on some parts of the bicycle, in particular if the chainstays are not adjustable. Furthermore, other user preferences may not be well-catered for by limited adjustability, for example frame stiffness, which is generally not adjustable. Moreover, the requirements can change with time, such as when the user becomes fatigued or due to changes in terrain—whilst current systems may allow a user to adjust one component when the user becomes aware of a general terrain change, such as an approaching downhill section, they do not allow for continual changes in response to detailed terrain features. Another limitation is that adjustments are made based on user-judgement, the accuracy of which can vary in dependence on user experience, luck and day-to-day experimentation.

WO 2006/034212 describes a system for dynamic adjustment of some vehicle parameters on the fly. User and terrain data can be input to the system, either as sensor data or previous user data and thus a geometry adjust function can be implemented. This function causes rotation of some components relative to each other, so as to provide a degree of position adjustment for the user, for example when riding a bicycle. However, any benefit is limited by the fact that the components themselves do not change, merely angles between some of the components.

It would be desirable to be able to adjust one or more components of a vehicle, for example during use of the vehicle, in a more systematic way based on knowledge of the rider and terrain.

### STATEMENTS

According to a first aspect of the present disclosure, there is provided a vehicle comprising: a frame component having an adjustable physical property; and a controller configured to receive data relating to the vehicle in use and, based on received data, determine a value of the physical property of the frame component.

The controller may be further configured to compare a current value of the physical property to the determined value, and if different, determine an adjustment of the frame component to cause it to have substantially the determined value. The controller may also be configured to generate instructions relating to the determined adjustment. The vehicle may further comprise an actuator arranged to adjust the frame component based on the instructions.

The received data may relate to properties of any one of: the vehicle; a terrain on which the vehicle is being used; a terrain ahead of the vehicle; a user of the vehicle; and data from another vehicle which is crossing the terrain generally ahead of the vehicle.

The data may be received in real-time and/or uploaded to the controller prior to use of the vehicle.

The vehicle may further comprise one or more sensors arranged to gather information during use of the vehicle, use the information to generate data and provide data to the controller in real-time. One or more of the sensors can be a terrain sensor, arranged to gather information about a terrain ahead of the vehicle. The terrain sensor may be arranged to gather information comprising one or more of: incline or decline; presence of an obstacle; smoothness; hardness; surface topography; surface friction and climate.

One or more of the sensors may be arranged to measure one or more parameters of the vehicle. The vehicle parameters may comprise one or more of: vehicle component lengths; forces sustained by vehicle components; angle between vehicle components; gearing selections; vehicle speed; vehicle acceleration; angle of vehicle relative to the horizontal in a direction of travel; tilt or roll angle of vehicle; angle of vehicle components; distance between vehicle components; forces applied to user interface components; speed of user interface components; tire pressures; and acceleration of user interface components.

One or more of the sensors may be arranged to measure one or more parameters of a user of the vehicle. The user parameters may comprise one or more of: heart rate; temperature; blood pressure; blood sugar levels; muscle fatigue; breath composition; position of the user relative to the vehicle; and angle of user body parts relative to the vehicle or each other.

The received data comprises one or more of: previously-generated terrain data pertaining to a terrain on which the vehicle is to travel; data from the vehicle or another vehicle captured from a previous travel across the terrain; vehicle component masses; vehicle component inertias; vehicle component compliances; suspension component settings; user mass; user segment inertias; user segment lengths; user gender; user fitness; user strength; user preferences for feel of vehicle; user preference for component stiffness; user preference for suspension hardness; user preference for vehicle dimension; and information input by the user.

The value of the physical property affects one or more of the following factors: comfort; handling; speed; controllability and efficiency of the user of the vehicle. The controller can be configured to determine the value of the physical property to optimize one or more of the factors in accordance with user demand.

In some examples, the physical property of the frame component may comprise a dimension of the component. The dimension may be adjustable by means of one or more of: an extensible portion; an elastic portion; the component having two parts overlapping to an adjustable degree; a mechanical fuse; an unfoldable portion; an additional portion that can be selectively incorporated into the component; the component being moveable relative another component; adjustment of an attachment of the component to another component; selective deployment of one of multiple alternative components; a rack and pinion; a worm gear; an inerter; and a travel limiter on the component.

In some examples, the physical property may comprise a compliance of the frame component. The compliance can be adjustable by means of one or more of: a chargeable portion; and a selectively deployable supporting portion.

The frame component may be one or more of: a supporting structural component; a rear triangle component; a front triangle component; a saddle post; a handlebar; a fork; and a wheel spacer.

According to a second aspect of the disclosure, there is provided a method comprising: receiving data relating to use of a vehicle; and determining, based on received real-time data, a value of a physical property of a frame component of the vehicle.

The method may further comprise comparing a current value of the physical property to the determined value and if different, determining an adjustment of the frame component to cause it to have substantially the determined value. The method may further comprising adjusting the frame component in accordance with the determined adjustment.

The received data can relate to properties of any one of the vehicle; a terrain on which the vehicle is being used; a terrain ahead of the vehicle; a user of the vehicle; and data from another vehicle which is crossing the terrain generally ahead of the vehicle.

The method may further comprise receiving some or all of the data in real-time and/or uploading some or all of the data prior to use of the vehicle.

The method can further comprise: gathering information during use of the vehicle; and using the information to generate data. In some implementations, gathering information may comprise gathering information about a terrain ahead of the vehicle. The information can comprise one or more of: incline or decline; presence of an obstacle; smoothness; hardness; surface topography; surface friction and climate. In some implementations, gathering information can comprise measuring one or more parameters of the vehicle. The vehicle parameters may comprise one or more of: vehicle component lengths; forces sustained by vehicle components; angle between vehicle components; gearing selections; vehicle speed; vehicle acceleration; angle of vehicle relative to the horizontal in a direction of travel; tilt or roll angle of vehicle; angle of vehicle components; distance between vehicle components; forces applied to user interface components; speed of user interface components; tire pressure; and acceleration of user interface components. In some implementations, gathering information may comprise measuring one or more parameters of a user of the vehicle. The user parameters can comprise one or more of: heart rate; temperature; blood pressure; blood sugar levels; muscle fatigue; breath composition; position of the user relative to the vehicle; and angle of user body parts relative to the vehicle or each other.

The received data can comprise one or more of: previously-generated terrain data pertaining to a terrain on which the vehicle is to travel; data from the vehicle or another vehicle captured from a previous travel across the terrain; vehicle component masses; vehicle component inertias; vehicle component compliances; suspension component settings; user mass; user segment inertias; user segment lengths; user gender; user fitness; user strength; user preferences for feel of vehicle; user preference for component stiffness; user preference for suspension hardness; user preference for vehicle dimension; and information input by the user.

The value of the physical property may affect one or more of the following factors: comfort; handling; speed; controllability and efficiency of the user of the vehicle. Determining the value of the physical property can comprise optimizing one or more of the factors in accordance with user demand.

The physical property of the structural component may comprise a dimension of the component. The dimension can be adjustable by means of one or more of: an extensible portion; an elastic portion; the component having two parts overlapping to an adjustable degree; a mechanical fuse; an unfoldable portion; an additional portion that can be selectively incorporated into the component; the component being moveable relative another component; adjustment of an attachment of the component to another component; selective deployment of one of multiple alternative components; a rack and pinion; a worm gear; an inerter; and a travel limiter on the component.

The physical property may comprise a compliance of the structural component. The compliance is adjustable by means of one or more of: a chargeable portion; and a selectively deployable supporting portion.

The frame component may be one or more of: a supporting structural component; a rear triangle component; a front triangle component; a saddle post; a handlebar; a fork; and a wheel spacer.

In relation to any of the vehicles or methods discussed above, the vehicle may be one of: a bicycle; a motorbike and/or sidecar; a moped; an all terrain vehicle (ATV); a skateboard; a pram; and a wheelchair. Other vehicles will occur to the skilled reader.

According to a third aspect of the present disclosure, there is provided a computer program product comprising a computer-readable medium storing instructions which, when executed by at least one programmable processor, cause the at least one programmable processor to perform operations comprising any of the methods discussed above.

According to a fourth aspect of the present disclosure, there is provided a control system for adjusting a structural component of a vehicle, comprising a computer-readable medium of the type discussed above and one or more actuators arranged to adjust the structural component based on the determined value.

Any of the above-discussed aspects may be applied to more than one component and more than one adjustable operating parameter.

### DRAWINGS

The present invention will now be described by way of example with reference to the accompanying drawings, in which:

FIG. 1 shows an exemplary vehicle;

FIGS. 2A-D show schematic views of components having adjustment mechanisms;

FIGS. 3A-F show further examples of rear suspension layouts to which the adjustment mechanisms of FIG. 2 could be applied;

FIG. 4A-C show exemplary sensor positions;

FIG. 5 shows a control system in accordance with some implementations; and

FIG. 6 shows a method in accordance with some implementations.

In the figures, like reference numerals indicate like parts.

### DETAILED DESCRIPTION

The current subject-matter relates to a vehicle which has one or more frame components that can be adjusted. For example, a dimension or compliance of the component may be adjustable. In some examples, the frame component is part of a load-bearing structure of the vehicle. The vehicle can have a control system which can receive data, which may include data generated from information gathered during use of the vehicle, and use that data to decide on an adjustment. Such adjustments may be made to optimize certain factors affecting use of the vehicle, such as performance metrics, for example handling or to allow the vehicle to travel faster or to allow greater efficiency of a user of the vehicle, for example the rider of a vehicle such as a bike. Such optimizations tend to be most effective for vehicles in which the user's body and how the user is using the vehicle has an influence on these factors.

Exemplary Vehicle—Bicycle

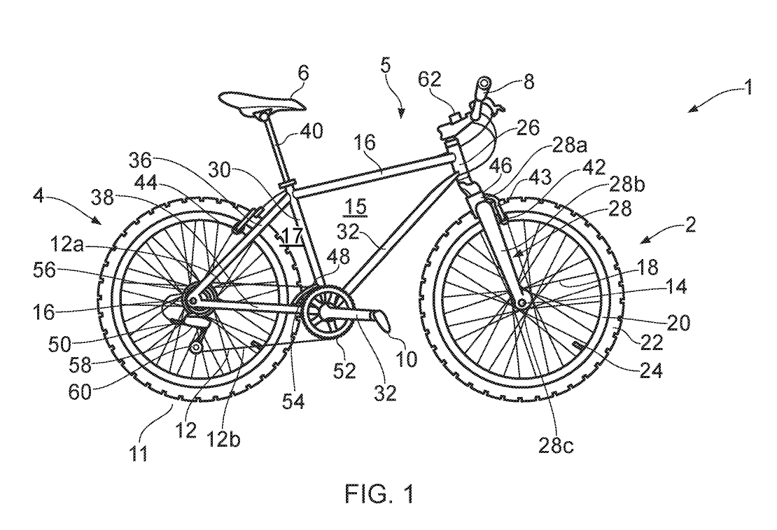

FIG. 1 shows a vehicle in accordance with examples of the present subject-matter. The exemplary vehicle is a bicycle 1 having a layout of basic components as known in the art. These are a front wheel 2 and a rear wheel 4, connected via a frame 5. Attached to the frame 5 is a saddle 6 on which a user can sit, handlebars 8 which a user can hold and pedals 10 on which a user can place his or her feet. The pedals 10 are connected via a chain 12 to the rear wheel 4, such that when a rider rotates the pedals 10, that causes the rear wheel 4 to rotate, thereby propelling the bicycle forwards along a surface 11 on which the wheels 2, 4 are resting. In the following, “front” and derivatives are used to designate various components of the bicycle 1 or surfaces thereof and to indicate the forward end of the bicycle 1 in the direction of travel. “Rear” and derivatives are used to designate various components and surfaces thereof and to indicate the rear end of the bicycle 1 in the direction of travel. “Upper” refers to components which are further away from a surface such as the ground on which the bicycle is disposed for use than “lower”, which indicates closer proximity to the ground. Similarly, “upper” may refer to a component surface facing generally away from the ground, whilst “lower” may refer to a component surface facing generally towards the ground. “Side” and “sideways” indicates surfaces, components or directions generally perpendicular to the direction of travel.

Components of the bicycle 1 will now be described in more detail.

The front and rear wheels 2, 4 in the exemplary bicycle 1 are substantially the same size, although this may not be the case. Thus their central hubs 14, 16 are arranged to be substantially the same height above a surface on which the wheels 2, 4 sit when the bicycle is at rest. The distance between the central hubs 14, 16 varies with any given bike, and depends, for example on the size of the frame 5. Standard bicycle wheels vary in size from a diameter of around 27-29 inches. A standard construction is to have multiple spokes 18 emanating from the hub, attaching at their distal ends to a circular rim 20, the spokes 18 and rim 20 being constructed of metal. A tire 22 surrounds the rim 20 and can be inflated with air via a valve 24. The tire 22 often contains an inner tube for holding the air under pressure, but this may be omitted in some constructions.

The frame 5 is formed of a number of components, which are generally elongate, tubular and welded together, but may be connected by other means or be integral with one another. The components of the frame 5 generally form a front triangle 15 and a rear triangle 17.

Components of the front triangle 15 are as follows. The uppermost component is a top tube 16, which is arranged to sit substantially horizontally or sloping at an acute angle upwards in the forwards direction relative to the surface 11. It will be appreciated that the surface 11 in FIG. 1 is flat, planar and horizontal, but that in reality the bicycle 1 may travel over a surface which is sloped and/or bumpy, in which case the angle of the top tube 16 to the surface may vary. The top tube 16 is connected to a head tube 26 at its front end, which is arranged substantially perpendicularly to the top tube 16, such that its end distal from the top tube 16 is connected to forks 28 which extend across each side face of the front wheel 2 to meet the ends of the hub 14. The forks 28 have a fork crown 28a, where they meet the top tube 16, two blades 28b that extend from the fork crown 28 across the faces of the front wheel 2, each blade 28b ending in a dropout 28c which attaches to the wheel hub 15. At its rear end, the top tube 16 is connected to a seat tube 30, which extends down to a bottom bracket (obscured in the figure) in which crank arms 32 can rotate. Each crank arm 32 is connected to a pedal 10. Also running from the bottom bracket to a position on the head tube 26 adjacent and below where the top tube 16 joins the head tube 26, is the down tube 32.

Components of the rear triangle 17 are as follows. Extending rearwards from a point on the seat tube 30 substantially at the same height at which the top tube 16 meets the seat tube 30 are a pair of seat stays 36 (only one visible in FIG. 1). The seat stays 36 extend across side faces of the rear wheel 4 to the hub 16 of the rear wheel 4. Running from the hub 16 of the rear wheel 4 to the bottom bracket is a pair of chain stays 38 (one of which is partially obscured in FIG. 1). In some examples, the seat stays 36 may be integral with the respective chain stays 38 disposed on the same side of the bicycle 1, each integral seat stay/chain stay forming a fork which fits over the hub 16 for attachment purposes. The third side of the rear triangle is the seat tube 30, common with the front triangle 15.

The frame 5 may be made from a variety of materials, for example steel, aluminium, titanium or carbon fiber. A standard way of denoting a frame size is by the length of the seat tube 30 and this can generally vary from around 13-21 inches. Commonly, the seat stays 36 and the chain stays 38 are smaller in cross-section than the seat tube 30, the top tube 16 and the down tube 32, but this is not essential. On some bicycles the seat stays are omitted altogether.

Slidably fitted into the seat stay 30 and projecting generally upwards therefrom is a seat post 40. The saddle 6 is fitted onto the seat post 40. The handlebars 8 are fitted on the top end (opposite to where it meets the forks 28) of the head tube 26.

A pair of front brakes 42 is attached via a caliper arrangement 43 such that the brake blocks are disposed either side of the rim 20. They can be operated by a lever on the handlebars, via the caliper arrangement 43, to bring them into contact with the rim 20 and thereby acting to stop rotation of the front wheel 2. Similarly, a pair of rear brakes 44 are attached to the seat stays 30 to stop rotation of the rear wheel 4. It will be appreciated that various types of brakes can be used, including disc brakes, which act on the wheel hubs 14, 16 rather than on the wheel rims.

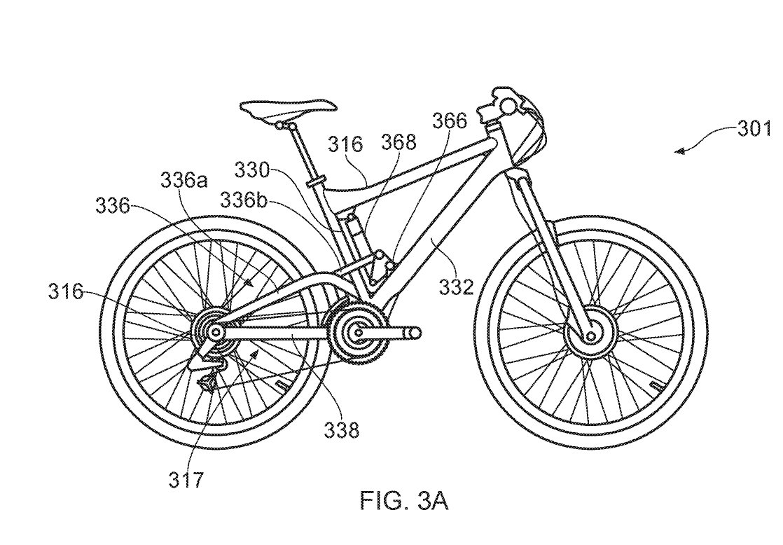

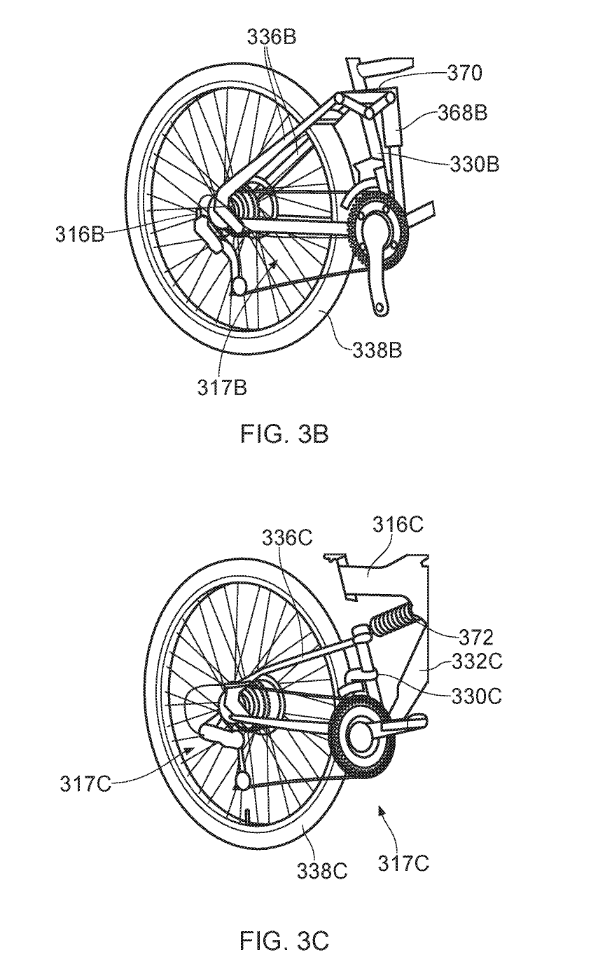

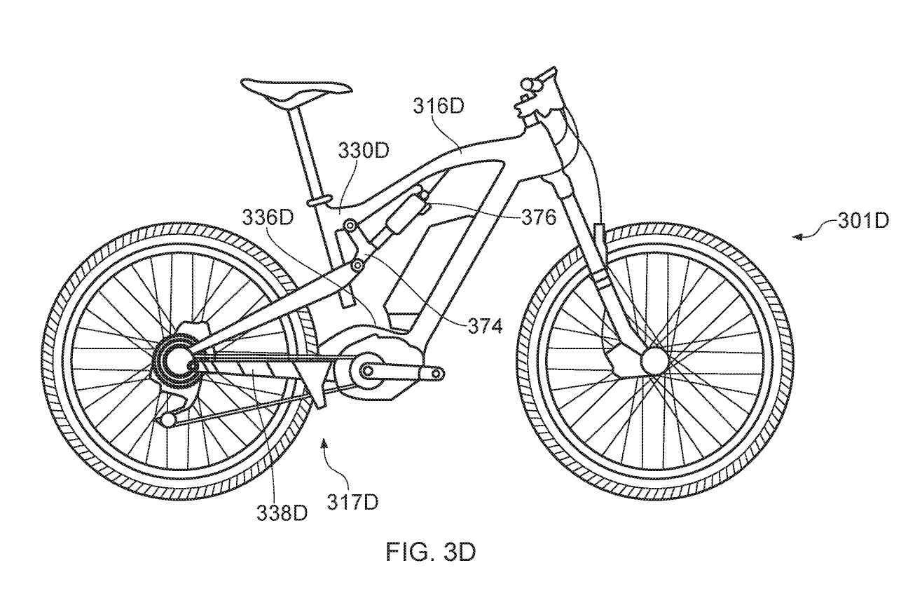

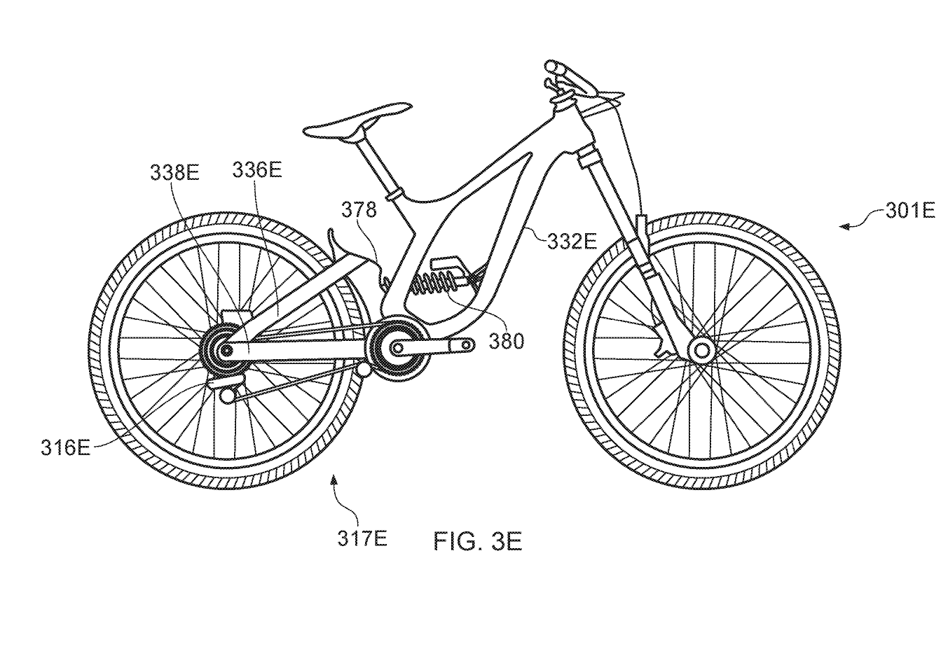

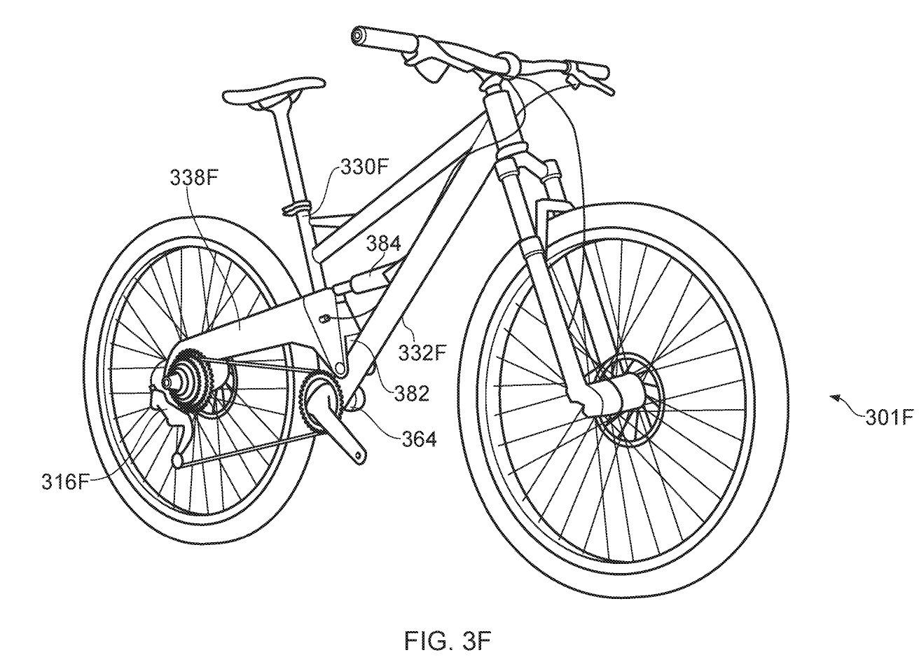

The bicycle 1 may be provided with a suspension system, which can comprise some or all of the following parts:
A front suspension may be provided by means of a shock absorber 46 in the front forks 28. This comprises two telescoping parts whose relative movement is controlled by a spring and a damper, one of which may be incorporated into each fork blade 28b or both of which may be in the fork crown 28a. The spring can be a metal or elastomer coil, or it can be provided by compressed air. The damper can be implemented using oil and shim stacks. It will be appreciated by those skilled in the art that other arrangements are possible.
A rear suspension may also be provided, to suspend the rear wheel 4 from the frame 5. Various arrangements are possible for the rear suspension, having a variety of pivots and levers or a flexible frame component. Some examples are discussed below with reference to FIG. 3.
The saddle 6 or the seat post 40 may be provided with a shock absorber, which on many existing bicycles is a spring only.
One or both wheel hubs can be provided with suspension.
One or more frame components may be provided with an integral portion having flexibility.

Finally, the bicycle 1 can be provided with a gearing system, although some bicycles are single-speed and thus have no gears. Various gearing systems will be familiar to those skilled in the art. One type is hub gearing. The exemplary bicycle 1 in FIG. 1 shows another type, namely a derailleur gear system. This includes a front derailleur 48 centred on the bottom bracket, and a rear derailleur 50 on the rear wheel hub 16. The chain 12 runs around the front and rear derailleurs 48, 50. The front derailleur 48 has a number of front sprockets 52 of different sizes, for example three sprockets, plus a front chain guide 54 which is operable to move the chain 12 between the sprockets 52. The front chain guide 54 is disposed on an upper portion 12a of the chain 12, near the front sprockets 52. The rear derailleur 50 also comprises multiple rear sprockets 56, a rear chain guide 58 and a chain tensioner 60. The rear chain guide 58 and the chain tensioner 60 are both disposed below the rear sprockets 56, in a lower portion 12b of the chain 12. In some examples, there may be anywhere between 5 and 10 rear sprockets 56. The rear sprockets 56 are generally smaller in diameter than the front sprockets 52. In operation, the rider can select a front sprocket 52 and a rear sprocket 56 in dependence on the desired gear ratio and the respective chain guides 54, 58 operate to slide the chain onto the selected sprockets. The bicycle 1 can be provided with lever shifters or twist shifters on the handlebars 8 to enable the rider to make the selections (not shown in FIG. 1). The shifters are connected to the chain guides 54, 58 by a cable or suchlike, as will be readily understood by the skilled person.

The exemplary measurements for wheel diameter and frame size set out above are for adult bicycles. It will be appreciated that smaller bicycles are available for children, which may have some or all of the above-described features and to which the examples described herein can be equally applied. Children's bicycles are typically sized according to wheel diameter rather than frame size and vary from about 12″ to 26″.

In accordance with examples of the present subject-matter, there is provided a controller 62, shown disposed on an uppermost surface of the handlebars 8, such that it is visually accessible and easily able to be touched by a rider of the bicycle 1. A purpose of the controller 62 is to control adjustments to components of the bicycle 1, as will be described in more detail in the following. The controller 62 may incorporate features of a cyclocomputer, which is used to measure and display information about the bicycle and a journey being ridden. For example, it can provide an indication of distance travelled, trip time, travel speed, pedalling speed and gear selected etc.

Performance and Adjustments

A number of the above-described components affect how the bicycle 1 operates and feels to its rider in use. In other words, the physical properties and characteristics of some of the components that form the structure of the bicycle 1 can make a difference to various performance factors or metrics. It may be advantageous to optimize one or more of these factors depending on the circumstances of use of a vehicle and the desired performance. The relative importance of each factor can depend on the user's characteristics and preferences, as well as the terrain on which the bicycle 1 is being ridden. Performance metrics and some components which can affect one or more of these factors and the effect of adjusting them are discussed in the following.

Some performance metrics which it may be desirable to control or optimize for are as follows:
Comfort or ride—this relates to how comfortable a user feels when cycling and can depend on how well the rider is cushioned from shocks. It may be paramount for some riders in some circumstances, but on the other hand, a given rider may be willing to sacrifice a degree of comfort in order to optimize other metrics. Pedalling speed is also a comfort factor and is affected by gear ratio, terrain, conditions e.g. wind speed and user strength.
Handling—this relates to how the bicycle performs in terms of directional stability and when required to change direction (cornering) or during acceleration/braking. In other words, different bicycles react differently to various forces that they are subjected to by the terrain on which they are being ridden and as a result of user demands. The less any adverse reaction, the better handling a bicycle is considered to have. For example, a bike that handles well maintains its direction despite being subjected to a braking force. Turning radius is also a handling characteristic, a smaller radius being considered to provide better handling because it is easier to effect a turn. With regard to cornering, inclination to oversteer or understeer is a poor handling characteristic.
Controllability—this relates to how easy or difficult it is for the rider to manipulate the bicycle to ride over a terrain. A bicycle that is responsive to user actions but which equally is designed to mitigate loss of control has good controllability. For example, a bike having good controllability would be responsive to the rider turning the handlebars but would equally be difficult for the rider to lose control of such as by turning the handlebars too far too quickly. If, on the other hand, it is hard for the rider to implement movements such as turning without losing control of the bicycle, the bicycle would be considered to have poor controllability. It may be important for the bicycle to be easily controlled, for example for a child's bicycle. A skilled rider, on the other hand, might not need the bicycle to have a good level of controllability because they possess the necessary skill to control movements themselves. Such a rider may prefer to sacrifice controllability for better handling by retaining a greater degree of control of the bicycle themselves.
Speed—a factor that may be important to some users is to be able to propel the bicycle as quickly as possible, for example when racing downhill. However, this metric may be incompatible with other metrics, for example good controllability.
Rider efficiency—certain characteristics of the bicycle affect the proportion of energy used by the rider's body that is converted to motion of the bicycle. Having a high efficiency may be important for a rider who is racing, cycling for long periods of time or for a fatigued rider.

Frame components of a vehicle such as the bicycle 1 have a number of physical characteristics. Such characteristics could be termed an internal or self-property. They may include properties such as a dimension (length, width or diameter/circumference), a mass, an inertia or a compliance. Although such properties may relate to a single component, adjusting such a property in one component can, in some cases, affect a relationship of that component with another component. For example, if the length of one of the three members of the front triangle is changed, that can in turn affect the relative angles between the three components or result in alteration to a suspension member. In many cases, whilst these physical characteristics are inherent to a component, they can nonetheless be adjustable and can be adjusted in accordance with some implementations of the current subject-matter. Some of the components of the bicycle 1 which can be adjusted and the effect of doing so are as follows:
Seat post 40—the length of this can be adjusted by moving it into or out of the seat stay 30, thus altering the length of the portion of it that projects out of the seat stay 30. Seat post length in turn affects saddle height relative to the pedals 10. Thus the seat post length can affect pedalling efficiency or comfort. As regards peddling efficiency, if it is too short and the saddle is thus relatively low for the rider, the rider will be pedalling in a cramped position and will not be able to exert as much force on the downward part of the pedal rotation as he or she would if they were able to extend their legs further. On the other hand, if it is too long and the saddle is thus relatively high for the rider, they might not be able to apply force for the full rotation of the pedals 10, due to not being able to “reach” properly. Thus it may be desirable to optimize the seat post length between these two extremes such that the rider can just extend their legs fully on the downwards motion. Having said that, rider preference may affect the optimal length, for example if the rider feels safer being seated lower such that they can reach the ground more easily, they may be willing to sacrifice some pedalling efficiency. One reason this might be the case is if riding downhill, because a lower saddle position lowers the combined centre of gravity of the rider and bike and can thus make it easier to control the bicycle, thereby preventing the rider losing control and crashing.
Masses and inertias of a supporting structural frame components—the centre of mass of any of the components of the frame 5 can be adjusted. This includes the top tube 16, the head tube 26, the down tube 32, the seat tube 30, the seat stays 36 or the chain stays 38. Such adjustments can affect the location of the centre of mass of the bicycle 1. The location of the centre of mass in turn affects handling by contributing to oversteer or understeer. One reason for this is that the location affects the degree of wheel flop i.e. the tendency of the front wheel to drop upon turning of the handlebars, thus leading to unexpected veering off curve. Wheel flop may be too high if the centre of mass is too far forward and thus the relative masses of the frame components can be adjusted to move the centre of mass and thus avoid excessive wheel flop.
Length of forks 28—this affects steering or head angle of the bicycle 1, which is the angle of the head tube (i.e. the steering axis or the axis about which the steering mechanism pivots) relative to the horizontal. The longer the fork length, the smaller the head angle. For example, it may be desirable for a mountain bike to have a lower or “slacker” head angle so as to improve handling on uneven terrain, especially for difficult descents. A typical head angle on a mountain bike is around 70°, which may be optimal for some descents, but not necessarily optimal for all descending gradients or for inclines.
Fork offset or rake—this is the perpendicular distance from the steering axis to the centre of front wheel. The exemplary bicycle 1 in FIG. 1 is shown as having a low fork offset. The fork offset can be increased by curving the forks at the dropout end 28c or adding a tab in that region or by mounting the forks at a smaller angle relative to the head angle. As an example, road racing bikes have a fork offset of 40-50 mm (1.6-2″).
Wheel size—the diameter of one or both wheels 2, 4 can be varied. If the wheel diameter is too small, the bicycle will be unstable, whereas if too large, its excessive mass will adversely affect handling. The head angle, fork offset and wheel diameter together affect trail. Trail is the distance between where the steering axis intersects the ground and where the front wheel contacts the ground. On most bikes, the distance is measured backwards i.e. the wheel touches the ground behind the steering axis. A higher trail results in greater stability, which is an indicator of good handling. However, a more skilled rider may prefer a lower trail or even a negative trail (wheel touching the ground forwards of the steering axis intersection) because although the bicycle may be less stable, it can be controlled better, especially along a difficult path. In terms of how each of the three parameters affects trail, a decrease in fork offset and/or a decrease in head angle and/or an increase in wheel diameter reduces trail and vice versa.
Lengths of supporting structural frame components—the length of any of the components of the frame 5 can be adjusted. This includes the top tube 16, the head tube 26, the down tube 32, the seat tube 30, the seat stays 36 or the chain stays 38. For example:
   the top tube length affects the distance between the saddle 6 and the handlebars 8. This can affect the angle at which the rider is sitting, which in turn affects their comfort (e.g. they may prefer to be more or less upright) and their efficiency (due to the angle at which they are directing force on the pedals and hence the relative usage of stronger and weaker muscles).
   The length of the seat tube 30, the down tube 32 and the seat stays 36 can affect the height of the bottom bracket above the ground, which can affect how long a seat tube is needed for a particular rider. For example, for a tall rider, it may be desirable to have a lower bottom bracket, so that an excessively long seat post can be avoided. This may be advantageous if the rider wishes to minimize how high above the handlebars he is sitting.
   It may desirable to be able to adjust the chain stay length in dependence on conditions. In particular, if the rear wheel is suspended, its centre will vary in height relative to the bottom bracket as the suspension compensates for uneven ground. On an incline or descent, when the rider is changing gear, the length of the chain stays can affect the forces experienced by the pedals 10 and chain 12 i.e. the motion ratio. These forces are also related to rider pedalling efficiency and controllability of the bicycle. Thus undesirable forces can be mitigated by adjusting the chain stay length accordingly.
   The lengths of the seat tube 30, the seat stays 36 and the top tube 32 can also affect the wheelbase—this is the distance between the ground contact points of the front and rear wheels 2, 4. This distance can affect handling and stability. For example, it can affect turning radius and angular inertia (which affects oversteer or understeer).
Compliance of frame components—this refers to the stiffness of the frame components. A very stiff frame results in less comfort because shock absorbance is less than with a more compliant frame. However, a stiff frame may be preferred for a racing bike because it can be more efficient due to less energy loss into the frame and because a skilled rider can control a stiffer-framed bike more easily. On the other hand, a road bike might require a more flexible frame so as to improve comfort and thus reduce rider fatigue. A mountain bike, though, should not have too compliant a frame because that would result in an uncomfortable feeling of excessive movement on bumpy terrain.
Suspension characteristics—whilst it may be possible to set a given suspension system at different settings, such variation is limited by the limitations of the suspension components, such as the maximum travel and the spring rate. Thus suspension components such as masses/inertias, spring curve, damping curve and maximum travel can be determined in dependence on rider preference and intended use of the bicycle and chosen accordingly.

Currently, bicycles of various geometries are available. For example, a bike with a given head angle can be selected for different types of mountain biking e.g. trails, cross country, timed descents, but cannot be varied during use, for example within any particular course. Examples of the invention can overcome such limitations, as will be explained below.

As indicated above, user characteristics can also have an effect on bicycle performance and on some or all of the above-described metrics. Examples of the presently-described subject-matter can take account of user characteristics. Some examples of such characteristics are:
Mass—the rider's mass can affect the centre of gravity and inertia of the bicycle and rider in combination, which can affect handling as explained with respect to frame components and masses. Rider segment masses can also have particular effects—for example, if the rider has a long, heavy body and likes to lean well forward when riding, this will have a greater effect in moving forward the centre of gravity than a rider with long legs and a petite frame who prefers to sit more upright.
Limb lengths—these can affect the requirements for frame component lengths and seat tube length and thus it may be desirable to take into account the rider's geometry when considering possible optimizations as described above. For example, a rider with a relatively short body and/or short arms might require a shorter seat tube as their “reach” to the handlebars 8 will be less than that of a rider with a longer body and/or longer arms.
Gender—an average woman is smaller than an average man and thus may prefer a frame size which is smaller overall. This, coupled with the rider mass, can affect the centre of gravity of the bicycle and can affect handling as explained with respect to frame components and masses. By way of another example, an average woman may have less upper body strength than an average man and therefore may prefer to be able to manipulate the bicycle with less force and hence prefer better controllability.
Riding position and fatigue—as mentioned previously, a rider's preferred position e.g. degree of leaning forward, height above the pedals 10 etc. can affect their requirements for some of the above-described bicycle adjustments. Moreover, as a rider becomes fatigued, their experience may be improved by different handling and control metrics. Component adjustments which optimize rider efficiency, for example in view of a reduced pedalling speed or rotational force applied to the pedals, may also be desirable. Moreover, as a rider tires, he or she may shift position, thereby shifting their centre of gravity, which may also necessitate component adjustments to optimize a desired metric.

None of the above lists is intended to be exhaustive and other metrics and possible adjustments may be considered.

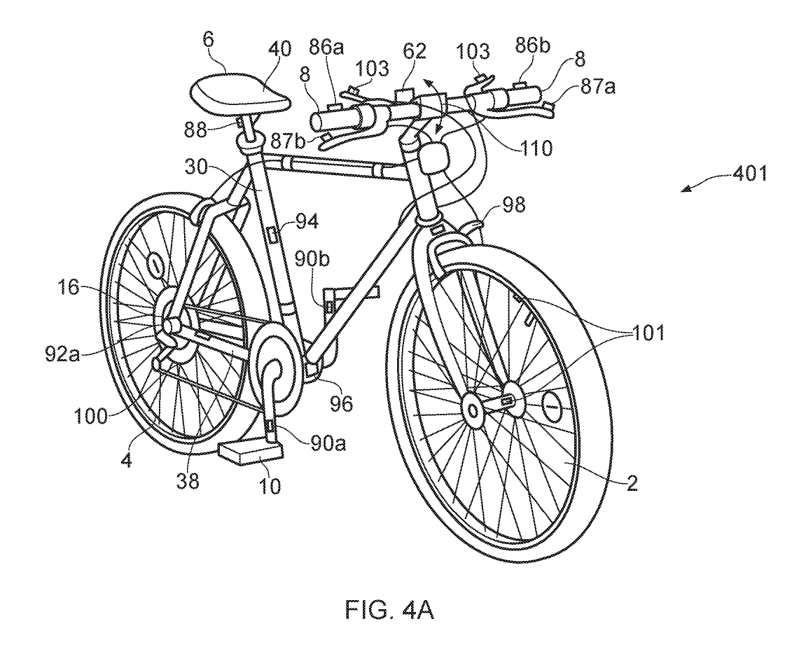

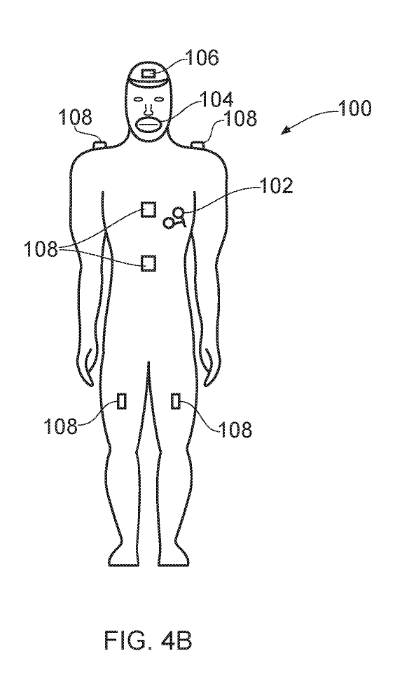

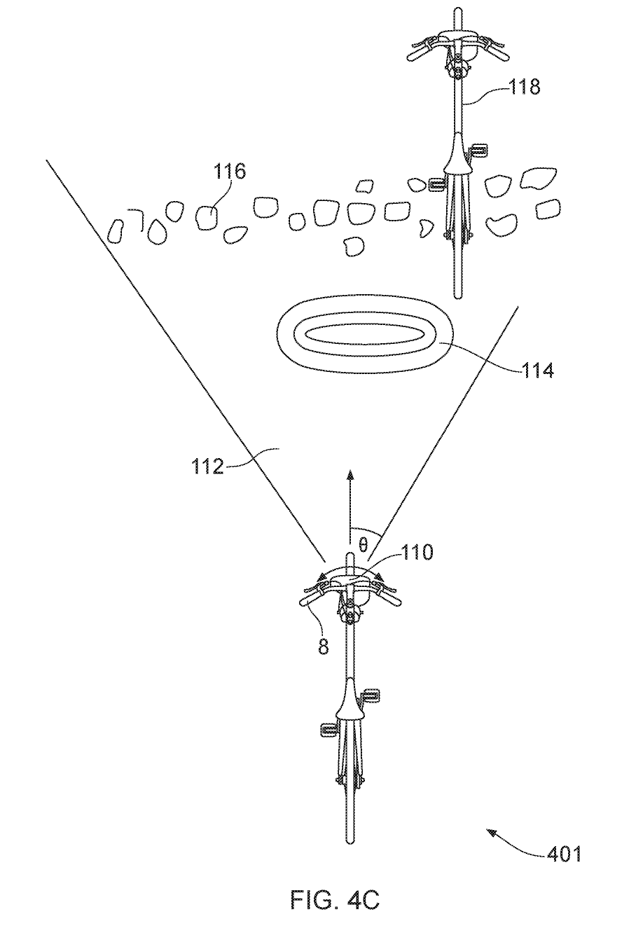

Finally, it should be noted that ongoing terrain changes can result in it being desirable to make adjustments to bicycle components. Such terrain changes can be determined by measurements of tilt of the bicycle and by scanning the upcoming terrain, as will be discussed with respect to FIG. 4 below. One example of a terrain characteristic that an adjustment may be useful for is an incline. A rider often naturally leans back when riding uphill, due to the effect of gravity on their body. This in turn affects the overall centre of mass of the bicycle and rider, which can result in instability and effects such as “bobbing”, where the front wheel lifts off the ground periodically due to rotational pedalling-induced forces. The present invention can reduce or eliminate such undesirable effects by calculating changes in the centre of gravity, and/or by taking account of other changes in rider position, and determining suitable component adjustments. For example, such adjustments could shift the centre of gravity back to a more desirable location which improves stability.

Examples of Adjustment Mechanisms

As described above, a length of a frame component of the bicycle 1, such as a load-bearing structural frame component, a seat post or a front fork, can be adjustable.

Various mechanisms can be used to cause any degree of adjustment desired, but in most examples of adjustment of a component length, an adjustment of up to around 5 cm or a couple of inches could be used to affect the above-discussed metrics to improve the rider's experience.

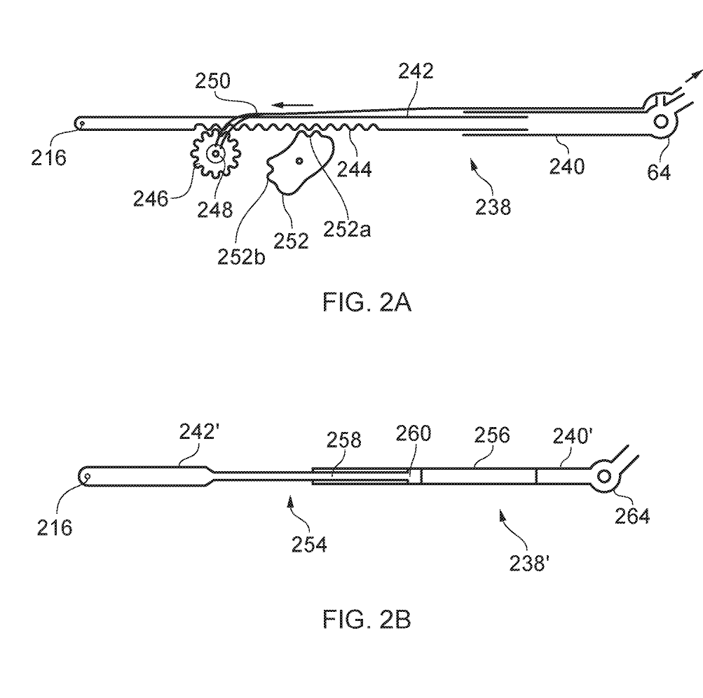

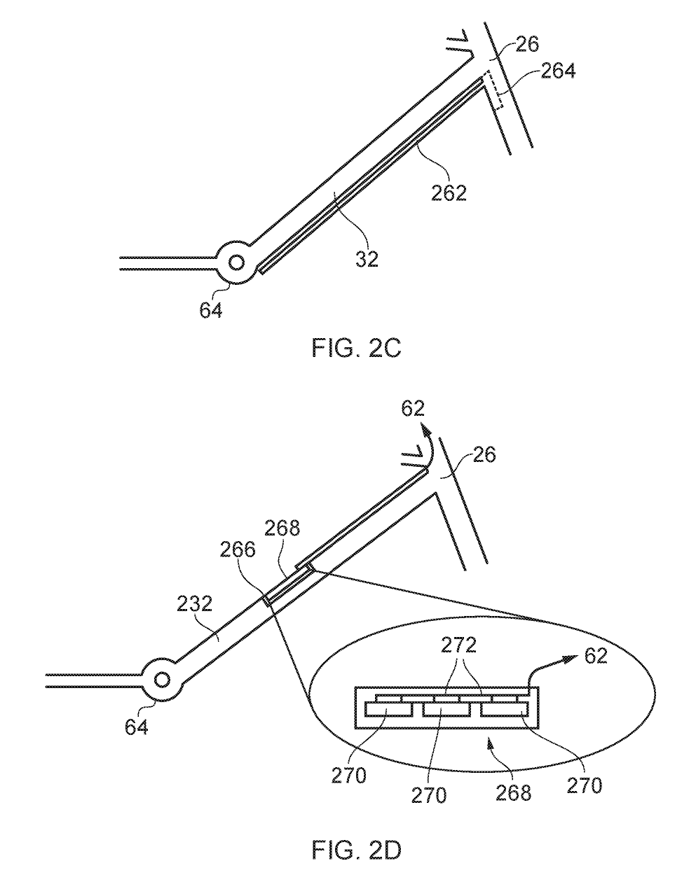

FIG. 2A shows an example of a mechanical adjustment for a frame component. This figure is shown schematically and not to scale; actual positions of the components could vary. For exemplary purposes, FIG. 2A shows a chain stay 238, but it will be appreciated that the same or a similar mechanism could be applied to other components. The chain stay 238 runs from the bottom bracket 64 (pedals and gears are omitted for clarity) and in this example is formed of two telescoping tubular parts. A first, outer tube 240 runs from the bottom bracket 64 and a second, inner tube 242 is held partially within the first outer tube 240 and extends to the rear wheel hub 216. Thus the length of the chain stay 238 is determined by the length of the outer tube 240 plus the length of the inner tube 242 which protrudes from the outer tube 240. Other components at the rear wheel hub, such as gears, are omitted for clarity. A section 244 of the inner tube 242 is toothed. Mounted for engagement with the toothed section 244 is a pinion gear 246 attached to an electric motor 248. The skilled person will appreciate that these could be mounted to the bottom bracket 64 or to the rear wheel hub 216, but they are shown towards the centre of the chain stay 238 for clarity. Wires 250 are shown as providing power to the electric motor 248—they are indicated as running along the tubes 240, 242 towards the bottom bracket 64 and onwards to the controller 62 (indicated by an arrow). A directional limiter 252 can also be provided, which is also positioned to engage with the toothed section 244.

In operation, power and instruction signals can be sent from the controller 62 via the wires 250 to the electric motor 248. Alternatively, the electric motor 248 could be powered by a separate battery. In dependence on the instructions received, the electric motor 248 can rotate either clockwise or counterclockwise. A clockwise rotation causes the pinion gear 246 to also rotate clockwise and thereby translate the inner tube 242 leftwards in the figure such that it moves further out of the outer tube 240 towards the rear wheel hub 216. Thus the proportion of the inner tube 242 held within and in overlap with the outer tube 240 reduces and consequently, the length of the chain stay 238 increases. A counterclockwise rotation of the electric motor 248 has the opposite effect, thereby decreasing the length of the chain stay 238. Other mechanisms such as spring arrangements, are possible.

Whilst the inner tube 242 should only move when the electric motor 248 rotates, the directional limiter 252 can be used as a safety feature to prevent movement of the inner tube 242 in one of the leftwards and rightwards directions. It has two sets of teeth 252a and 252b. In FIG. 2A it is shown in its position of maximum counterclockwise rotation. Due to the interaction of one set of its teeth 252a with the toothed section 244 of the inner tube 242, rightwards movement is prevented. It can rotate through around 90°, such that its other set of teeth 252b instead engage with the toothed section 244. The other set of teeth 252b is shaped to interact with the teeth of the toothed section 244 to prevent leftwards movement. The directional limiter 252 could be provided with a manual adjustment, such as a manually-operable switch to allow a user to rotate it to the desired one of the two positions. For example, if a rider knew he or she was about to enter a downhill section, the limiter 252 could be rotated to its clockwise-most position so as to prevent the chain stay 238 being increased in length. Alternatively, the limiter 252 could be controlled by the controller 62 via an electric motor or by a user operating the electric motor. Other locking and safety mechanisms will occur to the skilled reader.

As a variation on the arrangement of FIG. 2A, the electric motor 248 (and any electric motor used to operate the limiter 252) could be replaced with a solenoid valve(s). A solenoid valve could operate in response to instruction signals received from the controller 62. It could be positioned so as to act directly on the inner tube 242 to move it leftwards or rightwards. Alternatively, a rotatory solenoid could be provided, to rotate the pinion gear 246. Solenoid arrangements for causing multiple movements in one direction (linear or rotational) or continuous movements from multiple solenoid pulses such that a desired relative movement of the inner and outer tubes 240, 242 can be achieved, will be apparent to the skilled person.

FIG. 2B shows another example of a mechanical adjustment of a frame component. For comparison with the arrangement of FIG. 2A, it is shown applied to a chain stay 238′ but it could be applied to other frame components. The arrangement of FIG. 2B incorporates a gas spring 254 into the chain stay 238′. A first portion 240′ of the chain stay 238′, which is proximal to the bottom bracket 64, is connected to a cylinder 256 of the gas spring 254. A second portion 242′ of the chain stay 238′ is connected to a piston rod 258 of a piston 260 of the gas spring 254. Other features of the gas spring 254 will be known to the skilled person, but are omitted for clarity. It will be understood that the first portion 240′ of the chain stay 238′ could be integral with the cylinder 256 of the gas spring 254. It will be further understood that the second portion 242′ of the chain stay 238′ could be integral with the piston rod 258 of the piston 260 of the gas spring 254.

In operation, pressure inside the cylinder 256 can hold the piston 260 in position unless a force is applied to the piston rod 258. The position of the piston rod 258 relative to the cylinder 256 determines the length of the chain stay 238′. Thus the length of the chain stay 238′ can be adjusted when desired by application of a force to the piston rod 258. A lever or other user interface could be provided to allow manual application of such a force. Alternatively, the piston 258 could be controlled by the controller 62, which could control a force applied to the piston rod 258 by an actuator such as an electric motor or solenoid.

Other mechanisms to adjust a length of a component are possible. For example, other similar mechanisms such as a spring arrangement can be envisaged. Alternatively a mechanism such as a mechanical fuse could form a section of a component, which would “fail” upon application of a predetermined force, and thus increase in length. Other possibilities include rack and pinion systems, worm gears and inerters (known as J-dampers). Any suitable actuators could be used to operate any such mechanism.

It will be appreciated that any of the above-described length-adjustment mechanisms could be arranged differently in order to impart relative movement of telescoping tubes forming a chain stay. For example, arrangements which impart movement to the outer one of the telescoping tubes can be envisaged. Furthermore, inner and outer tubes of a chain stay 238, 238′ can be arranged in a reversed manner relative to the rear seat hub 216 and the bottom bracket 64. It will also be appreciated that any of the mechanisms could be configured to be operated under direct control by instructions from the controller 62 or alternatively by a mechanical actuator such as a switch, button or lever operated by a user such as a rider of the bicycle 1. It may be desirable to adjust a dimension other than the length of a component, for example a width or cross-sectional area.

As also mentioned above, another property of components of the bicycle 1 that can be adjusted is compliance. Various mechanisms can be used to cause a desired adjustment in flexibility.

FIG. 2C shows an example of a mechanism for allowing the compliance of a component to be adjusted. The component shown by way of example to demonstrate this mechanism is the down tube 32. An extra component in the form of a bar 262 is shown disposed underneath a section of the down tube 32, held by the head tube 26 within a slot 264 provided in part of the rearwards-facing region of the head tube 26. The slot 264 is longitudinal, extending along part of the length of the head tube 26, thereby enabling the position of the bar 262 relative to the down tube 32 to be adjusted. The bar 262 can be held in a desired location along the length of the slot 264 by any suitable means such as friction or an adjustable screw that can be deployed to hold the bar 262 or retracted to allow movement of the bar 262.

In operation, if it is desired to decrease the compliance of the down tube 32 i.e. to make it stiffer, the bar 262 can be moved upwards in the slot 264, towards the down tube 32, until it contacts the down tube 32. It can then be secured in this position such that the down tube 32 and the bar 262 are effectively integral. The presence of the bar 262 thus renders the down tube 32 stiffer because it increases its cross-sectional area. The bar 262 may also be made of a stiffer material than the down tube 32, for example steel, whilst the down tube 32 can be made of aluminium or carbon fibre, for example. If it is desired to increase the compliance of the down tube 32 i.e. to make it more flexible, the bar 262 can be moved downwards in the slot 264, away from the down tube 32. Thus when not in contact with the down tube 32, the bar 262 has no effect on compliance of the down tube 32. It can be secured in this position such that it is not contacting the down tube 32 or elsewhere around the circumference of the down tube 32.

It will be appreciated that variations on the arrangement of FIG. 2C are possible. For example, the bar 262 could be shorter if contact with just a portion of the down tube 32 would provide sufficient alteration of its compliance. Instead of being a single, unbendable piece of material, the bar could be formed of multiple parts which could be selectively unfolded such that together they could extend along different proportions of the length of the down tube 32. The bar 262 could be formed of a springy material or be a spring, and the down tube 32 and bar 262 could be provided with any suitable means to enable the bar 262 to be secured at a desired extension along the down tube 32. In respect of any of the above-discussed bars, multiple bars 262 could be provided, each of which could be moved into contact with the down tube 32 (or effective contact via contact with each other depending on their relative positions) to allow a number of discrete adjustments to compliance of the down tube 32. Any of the above-described bars could alternatively be adjustably secured at the bottom bracket end of the down tube 32 or indeed anywhere else along the length of the down tube 32.

The bar 262 could be manually actuatable or alternatively, could be moveable via direct control from the controller 62, for example by means of an electric motor or solenoid.

FIG. 2D shows an alternative example of a component of the bicycle 1 whose compliance can be adjusted. For exemplary purposes only, this example is described with reference to a down tube 232, but it could be applied to other components. Equally, the specific layout of components and their position within the down tube 232 and the number and shape of components is exemplary only. The down tube 232 has a recess 266 approximately halfway along its length, on its upper surface. In the recess 266 is set a damper 268. An enlarged, cross-sectional view of the damper 268 is provided in the figure. The damper 268 is generally cuboid, although it may be curved to fit with the contour of the down tube 232. The damper 268 is formed of a non-conductive material such as resin. Disposed within the resin and surrounded by the resin (such that the exterior of the damper 268 is non-conductive for functional and safety reasons) are three blocks 270 of piezoelectric material. Atop the blocks 270 and connecting them together is an electrode 272. The electrode 272 may be shaped to provide good connectivity to each block. The electrode 272 is connected to the controller 62. Such a damper is described more fully in U.S. Pat. No. 5,775,715 to Vandergrift.

In operation, the damper 268 can be used to adjust the compliance of the down tube 232 in one of two modes of operation. The first way is in a situation where the bicycle 1 is being ridden, for example, over rough terrain, resulting in forces on the down tube 232. The piezoelectric blocks 270 can absorb those forces and dissipate them, via the electrode 272, to the controller 62 (which may include a resistive power supply to absorb the resulting energy). Thus the damper 268 can reduce the compliance of the down tube 232 and can stop the bicycle 1 feeling too “bouncy”.

Alternatively, the second way is to supply power to the piezoelectric blocks 270 via the electrode 272. The blocks will deform, thereby providing a resistance to bending of the down tube 232 and effectively making it stiffer. In this way, the blocks 270 can absorb electrical energy and hold it as mechanical energy. A release of this energy can allow the blocks 270 and hence the down tube 232 to become less stiff. Supply of power and instructions to release stored energy can be implemented under control of the controller 62, in conjunction with a power supply or battery.

It will be understood that any of the above-described mechanisms for adjusting any property of components of the bicycle 1 can be applied to any of the front or rear triangle frame components, handlebars or seat post and that the above-described applications to particular components are exemplary only. Furthermore, any combination of adjustments could be used. For example, some components could have a mechanism for adjusting the length of the component, whilst other components could be provided with a mechanism for adjusting their compliance.

Equally, any component could be provided with mechanisms for adjusting both a dimension and compliance. Other mechanisms than those described above may be apparent to the skilled reader. It should also be noted that where control via wiring is described, such control signals could alternatively be provided wirelessly.

Alternative Bicycle Component Layouts

The bicycle 1 is shown as having a traditional rear triangle 17 in which the chain stays 38 are rigidly integral with the seat stays 36 in the form of a fork end for attachment in the centre of the rear wheel 4. It will be apparent that should a length of the seat stays 38 be adjusted as described above, in view of the otherwise rigid nature of the rear triangle 17, such an arrangement would necessitate an additional adjustment. For example, the length of the seat stays 36 could be correspondingly adjusted. Alternatively, the seat stays 36 could be attached in a pivoting manner at one or both ends, to allow absorption of the altered dimension of the chain stays 38 by their relative angles changing. In this case, and in layouts described below, the chain tensioner can absorb changes to some extent.

Some bicycles, however, have a layout that can allow the length of the chain stays to be adjusted without the need for a corresponding adjustment to another frame component. Some examples of such alternative layouts, to which examples of the present invention can be applied, are shown in FIG. 3. Some of these layouts may be used in bicycles intending for mountain biking, in view of the provision of rear wheel suspension in addition to the front wheel suspension of the bicycle 1 of FIG. 1.

FIG. 3 shows a bicycle 301 with an alternative rear triangle layout 317. The bicycle 301 has a pair of chain stays 338 (only one is visible in the figure). It also has a pair of seat stays 336 and a seat tube 330. Instead of meeting the seat tube 330 in a region where the top tube meets the seat tube 330, the seat stays 336 extend from a rear wheel hub 316 at a shallower angle to the horizontal than the seat stays 36 of FIG. 1. A first seat stay 336a (fully visible in the figure) meets a down tube 332 at its distal end in the region of the bottom bracket. A second seat stay 336b is joined to the down tube 332 a distance away from the bottom bracket (approximately a quarter of the way along the down tube 332 from the bottom bracket) by means of a pivoting joint 366. The pivoting joint 366 attaches to a top tube 316 via a damper 368. Thus the pivoting joint 366 can allow a change in angle of the seat stay 336b and the damper 368 can vary in length (for example by compressing or expanding within the limits of its travel) such that their combined geometry can adjust to accommodate a change in length of the chain stays 338.

FIG. 3B shows an alternative rear triangle layout 317B, which may be known as a linkage driven single pivot rear shock. The rear triangle 317B is generally formed of a pair of chain stays 338B, a pair of seat stays 336B and a seat tube 330B. The seat stays 336B extend from a rear wheel hub 316B at an angle more similar to that of the bicycle 1 of FIG. 1 than that of the seat stays 336 of FIG. 3A. However, rather than attach to the seat tube 330B, the ends of the seat stays 336B distal from the rear wheel hub 316B are pivotally attached to a respective bracket 370 (only one of which is visible in the figure). The brackets 370 are also pivotally attached to a damper 368B, the distal end of which is pivotally attached to the bottom bracket. Thus in a similar manner to the arrangement of FIG. 3A, the combined geometry of the pivoting attachments of the brackets 370 and the length of the damper 368B can alter to accommodate a change in length of the chain stays 338B.

FIG. 3C shows another alternative rear triangle layout 317C, which may be known as a single-pivot rear shock. The rear triangle 317C is generally formed of a pair of chain stays 338C, a pair of seat stays 336C and a seat tube 330C. However, the seat tube 330C is truncated close to where it is joined to the seat stays 336C, such that it is not connected to the saddle or a top tube 316C. There is provided a spring 372 between the seat tube 330C in a region corresponding to where it is joined to the seat stays 336C, but extending from the frontwards side. The spring 372 is attached to a meeting point of the top tube 316C and down tube 332C. Thus although the seat stays 336C can themselves be rigid and non-adjustable, a change in length of the chain stays 338C can be absorbed by compression or expansion of the spring 372, although the seat tube 330C may need to be pivotally attached.

FIG. 3D shows a bicycle 301D having another alternative rear triangle layout 317D. The rear triangle 317D is generally formed of a pair of chain stays 338D, a pair of seat stays 336D and a seat tube 330D. However, the seat stays 336D are not connected to the seat tube 330D as such, but rather, connect to a top tube 316D via a bracket 374, which is pivotally attached to both the seat stays 336D and the top tube 316D. A forward-most region of the bracket 374 is connected to a damper 376, which is pivotally attached at its other end to the top tube 316D at a location further forward than that at which the bracket 374 is attached. Thus if a length of the chain stays 338D were adjusted, the bracket 374 and the damper 376 would be able to absorb this movement accordingly.

FIG. 3E shows a bicycle 301E having another variation of rear triangle layout 317E. The rear triangle 317E is generally formed of a pair of chain stays 338E and a pair of seat stays 336E, along with an elongated bracket 378 which is pivotally attached to the seat stays 336E at their ends distal from a rear wheel hub 316E. The other end of the elongated bracket 378 is pivotally attached to a spring-damper 380, which in turn is attached at its other end to a down tube 332E. Thus the bicycle 301E has similar components to that of the bicycle 301D shown in FIG. 3D, with a different geometry that can absorb a change in length of the chain stays 338E in a similar manner.

FIG. 3F shows a bicycle 301F in which chain stays 338F double also as seat stays. The chain stays 338F are attached to a rear wheel hub 316F and extend towards a seat tube 330F. The chain stays 338F meet at their ends around the seat tube 330F in a single portion 382, which is shaped and sized so as to pivotally attach to either side of a down tube 332F by means of wings at a lower end, whilst pivotally attaching to a damper 384 at an upper end. The damper 384 is pivotally attached at its other end to the seat tube 332F at a point further from a bottom bracket 364 than the point at which the single portion 382 attaches to the seat tube 332F. Thus with an absence of separate seat stays and in view of the various pivotable joints and the damper 384, changes in length of the chain stays 338F can be accommodated.

The skilled reader will envisage many other variations of rear shock and rear triangle layout and will appreciate that adjustments in accordance with examples provided herein could also be used in such variations.

Data

In order to derive possible adjustments to components of a bicycle, various data can be input to the controller 62. This could be a mixture of fixed or predetermined data and information gathered by making measurements during use of the bicycle and provided to the controller 62 as data “on-the-fly” or in real time.

Measurements to generate data in real time during use of a bicycle can be made by using a number of sensors. Such sensors may make measurements pertaining to one or more of the bicycle, the rider and the terrain.

A perspective view of a bicycle 401 which is similar to the bicycle 1 of FIG. 1, or could have alternative layouts such as those shown in FIG. 3, is shown in FIG. 4A. For convenience, components of the bicycle 401 are labelled with the same reference numerals as those of the bicycle 1 of FIG. 1. FIG. 4A shows some examples of possible sensor positions to make measurements pertaining to a bicycle. It will be appreciated that the sensors are shown schematically as blocks in locations such that they are visible in the figure; the exact dimensions, shapes and locations of the sensors could vary from those shown. Examples of sensors that may be provided are as follows:
a) A pair of handlebar sensors 86a and 86b on each side of the handlebars 8. These can be disposed in a region in which a rider holds the handlebars 8. They can measure the force exerted on the handlebars 8 by the rider. This force might vary, for example, depending on the gradient of the ground on which the bicycle is being ridden, the force possibly increasing downhill and reducing uphill. Rider position can also cause a variation in force exerted on the handlebars 8. For example, such forces, especially low frequency forces, can be used to indicate the location or a change in location of the rider's centre of mass. In order to make such a calculation, the forces measured could be used in conjunction with forces at other rider-bike interface points such as at the seat (see b) below) and at the pedals (see c) below), along with knowledge of the orientation of the bicycle. The orientation could be determined by means of tilt sensors (see e) below).
b) One or more seat sensors 88 on the seat post 40. This can measure a force exerted by a weight of a rider on the saddle 6 and can also measure a direction of any force applied. Thus information obtained from such a sensor(s) can allow a determination of whether the rider is seated or standing and whether he/she is sitting straight on the saddle 6 or leaning forwards or backwards.
c) A pair of pedal sensors 90a and 90b on the pedals 10 to measure a force exerted by a rider on the pedals 10.
d) A pair of hub sensors 92a and 92b (not visible) on the rear wheel hub 16 to measure forces exerted on components of the rear wheel hub 16.
e) A tilt sensor 94 on the seat tube 30 to measure an angle to the vertical and/or the horizontal. For example, such a sensor could measure whether the bicycle 401 is upright or leaning to one side or another. It could also measure whether the bicycle is on flat ground or on a slope. Such a sensor could be implemented with an IMU (Inertial Measurement Unit)
f) A cadence measurement device 96 mounted in the region of the pedals 10, which can measure the rotational speed of the pedals 10.
g) A suspension sensor 98 on the front forks 28 for measuring the front damper setting and/or movements. Similar sensors could be provided in any rear suspension system.
h) A chain sensor 100 mounted to measure chain tension. Such a sensor can take the form of a magnetic pick-up mounted to a chain stay 38 or on a chain cover if one is provided.
i) A wheel speed sensor 101 mounted on a wheel hub, to pick up wheel speed. A corresponding part can be provided on the interior face of a wheel 2, 4. FIG. 4A indicates a wheel speed sensor 101 on the front wheel 2, but one could be provided instead or as well on the rear wheel 4.
j) One or more gear sensors 103 to detect the current gear wheels selected and hence a gear ratio. A gear sensor 103 could be connected to each gear adjustment shifter to thereby detect gear changes. Alternatively, a position sensor could be put on the derailleur, for example on a chain guide, to determine the selected crank wheel(s).
k) A pair of brake sensors 87a and 87b provided on each brake lever. Alternatively or additionally, force present on the brake blocks could additionally be measured, as could hydraulic brake line pressure.

The sensors can be a mixture of accelerometers, magnetometers and gyroscopes. For example, sensors a) through d), g), h) and k) could be accelerometers to measure force. Sensors f) and i) could be a magnetometer which can detect rotation by “picking up” a signal each time a corresponding sensor part on the moving bicycle part rotates across the sensor. Sensor e) could be a gyroscope which can measure angular velocity and hence a degree of tilt.

At least some of the above sensors or other similar sensors mounted on e.g. handlebars, bottom bracket, rear wheel hub, crankset may provide useful information about the bicycle, for example by facilitating determination of undue forces on the hub or chain which indicate the need for an adjustment of the bicycle. Furthermore, at least some of the same sensors can be used to provide information about the rider, and thereby determine the rider's power output. The accelerations and hence forces measured by accelerometers can be combined with the speed of the bicycle (measured, for example, by a cadence sensor) to calculate power delivered to the bicycle by the rider.

FIG. 4B shows examples of possible locations for sensors on a rider 100 to measure data pertaining to the rider. Some examples are:
a) A heartrate monitor 102 to measure heart rate. Alternatively, a similar sensor could be placed on a pulse point.
b) A breath composition sensor 104, shown attached by means of a mask for exemplary purposes. Breath composition provides an indication of the efficiency of a person when exercising by measuring the volume of air inhaled and the concentrations of oxygen and carbon dioxide in the inhaled and exhaled air. If a rider undergoes strenuous enough exercise, these measurements can be used to calculate the person's VO₂ max (maximal oxygen consumption/peak oxygen uptake in litres/min of oxygen), which reflects their cardiovascular fitness.
c) One or more sensors 106 mounted in or on the rider's helmet. Such a sensor could, for example, be a tilt sensor, which could be used to determine the angle at which the rider is holding his or her head, which can indicate the incline on which the bicycle is being ridden and/or rider fatigue.
d) One or more sensors 108 mounted on parts of the rider's body, such as legs, arms, shoulders, upper or lower back. Such sensors could be tilt sensors, which would determine the angle of the body part on which they are situated. The angle of a body part might indicate optimal or sub-optimal riding position. Measurements from multiple such sensors could be combined to provide an overall picture of rider position.

Other measurements could be taken such as rider temperature, blood pressure, blood sugar level and muscle fatigue. All or selected ones of the measurements could be taken into account when determining suspension settings. For example, a sub-optimal position could indicate fatigue and/or that the rider is experiencing discomfort and an adjustment could be made which also takes account of the rider's cardiovascular fitness.

FIG. 4C shows a top view of the bicycle 401. Indicated substantially at the centre widthwise and on the front of the handlebars 8 is a terrain sensor, such as a terrain scanner 110 for detecting the terrain ahead of the bicycle 401. The terrain scanner 110 is also indicated on FIG. 4A. FIG. 4A further shows that the terrain scanner 110 or a component thereof can rotate in a generally vertical arc, as indicated by a double-ended arrow. FIG. 4C shows that the terrain scanner 110 or a component thereof can also rotate in a generally horizontal arc. Thus in a horizontal (azimuth) plane, the terrain scanner 110 can detect the terrain ahead within a forthcoming triangular area, the boundaries of which are determined by an angle θ either side of the direction of travel through which the terrain scanner 100 is able to take measurements. In some implementations, the value of 8 may be chosen to be narrower to reduce data bandwidth requirements. In other implementations it may be chosen to be larger to allow scanning terrain into which the rider may decide to turn. In a vertical (elevation) plane, it may be convenient to set the terrain scanner 100 such that it is able to take measurements of the ground and through a range of angles at least up to and in some cases beyond an angle to the horizontal/ground at which the bicycle 401 is disposed. Thus the terrain scanner 100 can detect forthcoming disturbances or changes in the ground such as gradient changes, holes, unavoidable bumps such as a bump indicated by reference numeral 114 and rough sections such as rocks 116. The terrain scanner can also detect forthcoming obstacles such as another bicycle 118 or trees, people and other vehicles. Information on atmospheric and weather conditions including precipitation, wind speed and light levels could also be gathered. Thus a wide variety of terrain information can be gauged in a region generally ahead of the bicycle 401.

The terrain scanner 110 can be a separate device from the controller 62 or the two can be implemented as a single device. In implementations where they are separate devices, they can be arranged to communicate with each other, either by wire or wirelessly. Thus the controller 62 can receive data from the terrain scanner 110. Data gathered could be processed either in the terrain scanner 110 or in the controller 62 or part-processed in each device.

The terrain scanner 110 can be implemented by means of a Light Detecting and Ranging (LIDAR) sensor. A LIDAR sensor uses a laser light to scan the terrain. The light can be shone ahead and a time taken for the light to be reflected and return to the scanner 110 can indicate the presence of an object and how far away the object is from the scanner 110. Various suitable LIDAR sensors will occur to the skilled reader. For example, for implementations of the present invention, an eye-safe laser wavelength and power combination might be chosen, for the safety of people in the vicinity of the bicycle 401. The selection of laser parameters may also depend on whether objects and/or atmospheric conditions are to be detected. As the terrain scanner 100 is to be used on a moving vehicle, light pulse frequency and thus data collection speed can be chosen to be sufficient so as to allow the processing of the data quickly enough to warn the user of the terrain ahead and/or to make adjustments to the bicycle 401. Equally, a detector that can pick up signals at a corresponding rate can be chosen. The terrain scanner 110 can be implemented by two LIDAR sensors or an array of sensors. A dual oscillating mirror within the terrain scanner 110 can be used to achieve the required azimuth and elevation detection ranges. The terrain scanner 110 may also incorporate a GPS (Global Positioning System) and an IMU to allow determination of absolute position of the bicycle, which may be useful for safety reasons or to determine if competitors in a cycling competition have diverted off the specified route, for example.

The terrain scanner 110 could be implemented by means other than a LIDAR sensor. One possibility would be to use a graphical method, implemented by a camera capturing images which could be computer-analysed. Such a system could work by building up a point-cloud of distances from multiple points to the camera, which could be used to determine the 3D terrain ahead. The image capture could be repeated at time intervals sufficiently small in comparison to the speed of the bicycle 401. Another possibility would be an optical flow sensor, using an image correlation technique which compares images captured by a camera of the terrain ahead with known images. A known image could be, for example, that of a flat surface; thus obstacles would be detected in view of the fact that they would produce a different image than the known one.

In addition to detecting the presence of another bicycle such as the bicycle 118, it may be useful for the bicycle 110 to be able to receive feedback on the forthcoming terrain from the bicycle 118. The bicycle 118 could be provided with its own terrain sensor and/or component sensors for sensing impact of the terrain where the vehicle is located, as well as a sensor for looking ahead of the bicycle 118, that could provide its detected data to the terrain scanner 110 or directly to the controller 62 or to another detection device on the bicycle 401. In this way, the controller can also take into account terrain information further ahead than the terrain scanner 110 can readily detect. For example, the bicycle 118 may arrive at a sudden decline before the bicycle 401 that is not “visible” to the scanner 110. Thus the controller 62 can receive an earlier indication of the decline than would be possible by receiving data from the terrain scanner 110 only. The controller 62 can receive such data from multiple bicycles at different locations ahead of the bicycle 401.

Those skilled in the art will appreciate that measurements from the various sensors can be combined or triangulated. Furthermore, where wired connections are mentioned above, such connections could alternatively be provided wirelessly.

Other, pre-known or pre-determined data can be provided to the controller 62 for use in determining possible adjustments to the bicycle 1, 401. Some examples of such data are:
previously-generated terrain data—for example, the bicycle may be being ridden over a known race course whose surface topography and inclines can be measured or are known in advance, for example following a previous outing on the terrain made by the bicycle or another bicycle.
masses of bicycle components and thus centre of gravity
inertias of bicycle components
initial component compliances
suspension component settings (e.g. sag) and maximum travel e.g. of a damper
mass of the rider
rider body part (segment) inertias and/or lengths
rider gender/preference of bicycle dimension
rider fitness, including previously-determined VO₂ max
rider preferences for the feel of the bicycle and hence a range of preferred component stiffness and/or suspension hardness.
other information input by the rider such as fatigue levels, health condition, preferred pedaling rotation speed range
tire rim width and tread configuration of the front and rear wheels
relationship between force applied at the brake levers and force resulting at the brake blocks and consequent speed reduction of the wheels and hence the bicycle

Control System

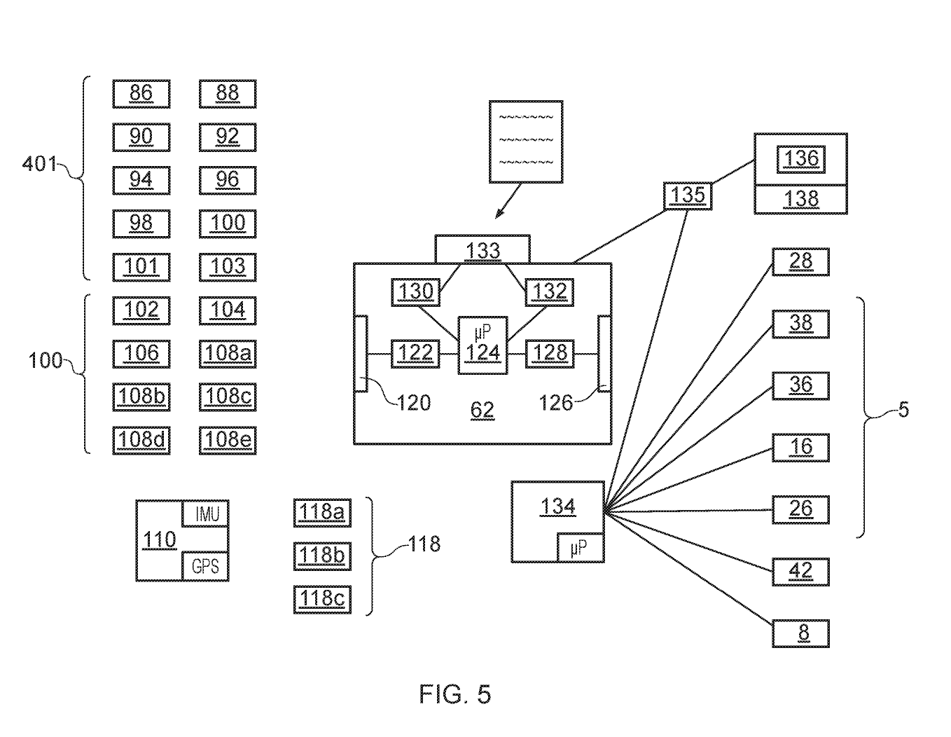

FIG. 5 shows schematically an exemplary control system 500 which can be used in some implementations. It will be appreciated that, whilst various functions of the control system 500 are shown as separate from or integral with other functions, various of the functions could be implemented together or separately and in hardware or software or a combination thereof. The elements are shown as corresponding to the elements discussed above, but the number, type and position of sensors and components could vary from that shown. It will be understood that it is not a requirement for any given bicycle or rider to be provided with all the sensors shown, but rather it is possible that selected ones of the exemplary sensors are present.

The sensors provided on the bicycle 401 as discussed with respect to FIG. 4A are indicated (one box indicating a pair of sensors on left- and right-side components where applicable). The sensors provided on the rider 100 are also indicated. The terrain scanner 110 is also indicated and can include an IMU and a GPS. An exemplary number of other bicycles 118 are indicated, the boxes representing sensors/data transmitting devices on such bicycles. To maintain clarity of the figure, all these sensors/transmitters are shown as wirelessly connected to the controller 62, but they could alternatively be connected by wire.

The controller 62 can include a receiver 120, which can connect via a segmentation engine 122 to a microprocessor 124. The microprocessor 124 can also connect to a transmitter 126 via a reassembly engine 128. The microprocessor 124 can also communicate with a RAM 130 and a ROM 132. The controller 62 has a data input device 133, which may be a USB port or a user interface such as a keyboard or touch screen. The latter may be used to select a performance metric setting (see below). A power supply 134 is shown external of and connected to the controller 62, although the two may be integral. The power supply may have its own microprocessor so that it can process instructions and deliver power accordingly. The microprocessor 124 and the power supply 134 are each connected via a display driver 135 to a display 136 which has a user interface 138. The power supply 134 is also connected to the various components which could be adjusted as discussed above. It will be understood that these connections are intended to indicate a connection to one or more actuators associated with one or more mechanisms for adjusting each component.

In operation, the sensors on the bicycle 401, the sensors on the rider 100, the terrain scanner 110 and/or those on the bicycles 118 can gather data “on-the-fly” during use of the bicycle 401. They can send the gathered data to the receiver 120 of the controller 62, which can segment it so as to provide data in coherent blocks, each pertaining to one sensor, to the microprocessor 124. The microprocessor 124 can also receive data from the RAM 130. The data sent by the RAM 130 can be any of the pre-known or pre-determined data discussed above which relates to the bicycle when in use. This can be input during use of the bicycle or prior to use or both. As well as detailed data specific to a rider or a terrain that the rider plans to use the bicycle on, it could also take the form of a selection from a number of performance metric settings e.g. “optimize handling”; “optimize speed”; or “downhill racing”. The microprocessor 124 can also receive information from the ROM 132, such as programs which may include mathematical equations such as equations of motion. These could be uploaded to the ROM 132 during manufacture of the controller 62 or they could be input and/or updated via the user interface 133. The programs and equations can be used in processing the data received from the RAM 130 and the receiver 120 to determine a value of a physical property of one or more of the components of the frame 5, the handlebars 8, the forks 28 or the seat post 40, such as length or compliance. This calculation can aim to determine an optimal value for such physical properties, for example to deliver a selected performance setting or to prevent the rider losing control of the bicycle on difficult terrain. For example, it may be that a particular location of the centre of gravity of the rider and bicycle combined or a particular trail length would be advisable, and the calculation could determine what the optimum position would be and then decide on the value of one or more physical properties of the component(s) that would achieve that. The microprocessor 124 could also make other calculations such as to determine appropriate suspension settings or gear ratio for example. All the data received by the microprocessor 124 could be used in any given calculation or alternatively, the microprocessor could select some data to be used.

Based on data gathered from the various sensors previously to a current time, or historically, the microprocessor 124 will also know the current value of the physical properties of the various adjustable components at the current time. The ROM may also store base (default) settings for component lengths, masses and inertias, which may be used as initial settings prior to use of the bicycle 401. The microprocessor can thus determine whether each decided value matches its respective current value and if not, determine an adjustment required to achieve the decided value.

Having determined what adjustment(s) to which component(s) would be desirable, the microprocessor can then send that information to the reassembly engine 128 for reassembly into instruction blocks each pertaining to an adjustable component. The reassembly engine 128 can then deliver the reassembled instructions to the transmitter 126, which can transmit the instructions to the display 136 via the display driver 135. Some further processing of the instructions may occur, for example in the display driver 135, prior to display so as to render them human-readable.

In some implementations, the rider 100 could then read instructions as to what adjustment(s) to make to what component(s) and decide whether to implement the adjustments shown. Adjustments could be effected by means of user-operable actuators, such as the ones discussed above with respect to FIG. 2. A suggested gear ratio could be displayed, optionally along with which cogwheel numbers to select to achieve that ratio.

In other implementations, the controller 62 could implement the adjustments itself by sending appropriate instructions to the power supply 134, which would process them further as necessary such that it could then provide appropriate power and control instructions to implement the adjustment(s). Alternatively, the power supply 134 could merely provide pulses of power as directed by the instructions, whilst the microprocessor could send instructions directly to the component actuators as to an adjustment(s) to be made. In some implementations, the rider 100 may be asked to take some action too, for example with regard to the adjustment mechanism of FIG. 2A, the rider may be asked to rotate the directional limiter 252 such that a length adjustment opposite to that of a previous adjustment can be made (i.e. longer rather than shorter or vice versa). Equally, the display 136 could be used to warn the rider that adjustment(s) are about to be made so that he or she is not taken by surprise when e.g. the saddle 6 lowers.

Thus it will be appreciated that any of the adjustment mechanisms could receive instructions either directly from the controller 62 or indirectly by way of the controller 62 displaying a suggested adjustment or setting to the rider and the rider actuating an adjustment mechanism accordingly. Thus the controller 62 can cause implementation of an adjustment either directly or indirectly.

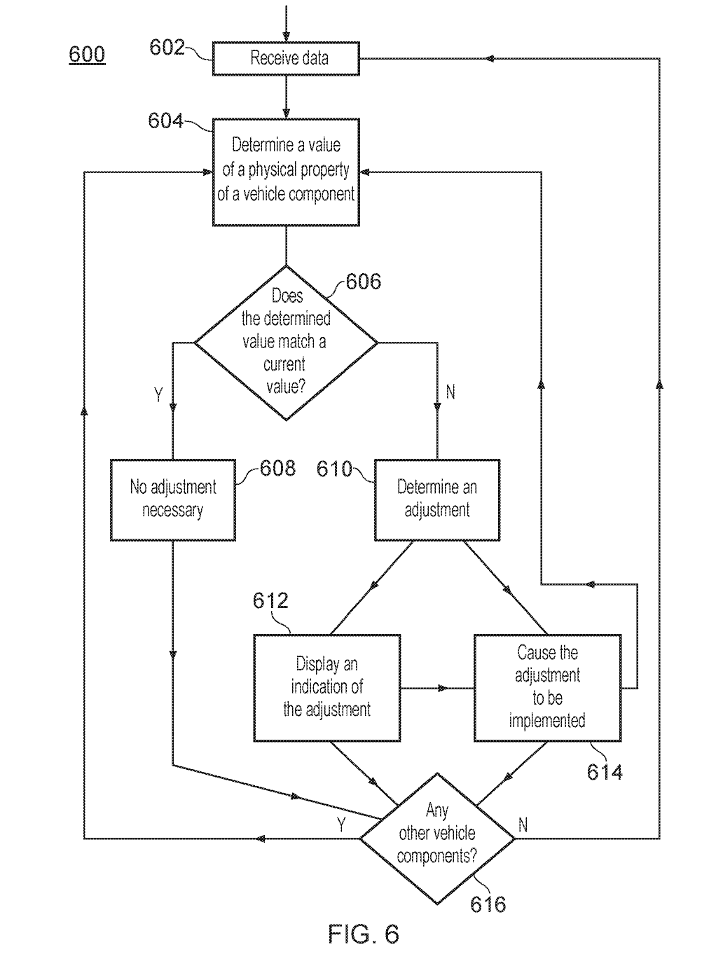

FIG. 6 shows a process flowchart of a method in accordance with some implementations of the present subject-matter. The method of FIG. 6 can be applied to a bicycle or to other vehicles, for example those discussed below.

At 602, data is received. This could be various types of data, and could be received prior to use of the vehicle or from measurements made during use of the vehicle or both. Some examples are discussed above. The data can be received at the vehicle, for example at a control device such as the controller 62.

At 604, the controller or other device that received the data, or another device that the receiving device has forwarded the data onto, determines a value of a physical property of a frame component of the vehicle. The value can pertain to a dimension of the component, such as a length or width, or to another property such as compliance. This determination may take account of current or previous values of the same or other components of the vehicle. As discussed above with respect to FIG. 5, the device may select some of the received data to determine the value.

At 606, a microprocessor in the controller or other device can compare the determined value to a current value of the physical property and hence whether the determined value matches or is equal to the current value. The term “matches” can include an exact match or substantially matching, such as within a small tolerance. For example, if a change in length of the component of 1 inch (2.5 cm) were required to make a material difference to a given performance metric, the controller could deem that a difference of less than ¼ inch (approx. ½ cm) constitutes a match.

If a match is found, at 608, no adjustment is necessary and the method proceeds to 616. If the determined value is found not to be equal to the current value, at 610, an adjustment is determined. An indication of the adjustment may be displayed at 612. At 614, either before, simultaneously with or after 612, the microprocessor 124 can cause the adjustment to be implemented. As discussed above, this could be by a user following displayed instructions or by the microprocessor 124 sending instructions to an actuator associated with the component to be adjusted and/or by control of a power supply such as the power supply 134 to act on an adjustment mechanism associated with the component, such as one of the adjustment mechanisms shown in FIG. 2. Any adjustment made may result in the value of the property of the component being equal to or substantially equal to the calculated desired value. Substantially equal can include an adjustment that renders the value closer to the desired value.

Following 608 or 612 and/or 614, it is determined at 616 whether any other components should be assessed for possible adjustment. For example, if a seat post length has been adjusted, it may be decided to adjust a front fork length too, so as to maintain stability. If the answer is no, the method can resume at 602 upon receipt of further data, for example at a next time period. Thus the method can repeat at each next time period for a duration of use of the vehicle. If the answer is yes, the method returns to 604, where a value of a physical property of another component is determined. Once all components which are being considered for adjustment have been assessed by the method commencing at 604, 616 will return a negative answer and the method can resume at 602.

Other Vehicles

Some examples discussed above used a bicycle as an exemplary vehicle to which the principles of the invention can be applied. Similar principles can be employed to optimize other vehicles for particular performance metrics, taking account of the terrain over which such vehicles are travelling and in some examples, adjusting other components than the bicycle components discussed above. Some other exemplary vehicles include:
A bicycle with child's trailer (tag along) or child's bike attached—as well as having adjustable components per a bicycle alone as previously discussed, suitable sensors to make measurements of the trailer or child's bike could additionally be employed. For example, the addition of a child on a trailer or bike shifts the centre of gravity of the vehicle as a whole and it may be advantageous to adjust the trailer or child's bike instead of or as well as the bicycle to which it is attached, perhaps to improve handling. A trailer or child's bike has a tendency to tilt more than the bicycle to which it is attached, especially when stopping, starting or cornering, and it may be possible to mitigate the adverse effect of this on an adult rider of the bicycle, by optimizing for handling. As another example, the centre of gravity can be towards the rear of the vehicle, which presents a risk of tipping over backwards when on an incline, especially if starting up on an incline—some implementations of the current subject-matter could be used to reduce this risk.
Motorbike—the geometry of a motorbike can affect its performance in a similar way to that of a bicycle. In particular, the fork angle has an effect on handling capabilities and could be adjusted in some implementations of the present subject-matter by, for example, increasing or decreasing the length of the fork. Some other effects, such as terrain change reactions, may also be usefully taken account of. For example, a change in components so as to take account of the rider shifting position on an incline or decline may be desirable. However, such adjustments may be less useful than when made in respect of a bicycle, due to the rider weight and position having less influence in the characteristics and performance of a heavier, engine-powered vehicle. The control system could be programmed accordingly, perhaps to make lesser adjustments in view of slope than would be made for a bicycle.
Motorbike with Sidecar—in this case, the “user” could refer to a rider of a motorbike, or an occupant of a sidecar of a motorbike. In a somewhat similar manner to a child's trailer, a sidecar can have an effect on the centre of gravity of the vehicle as a whole, which can present stability problems during cornering for example. Thus implementations of the present subject-matter could mitigate such difficulties by taking measurements on the sidecar as well as on the motorbike and using data pertaining to both the rider and the sidecar occupant, optionally making adjustments to one or both of the sidecar and the motorbike. For example, the sidecar wheels could be on runners such that the wheelbase could be adjusted in some circumstances.
Moped—considerations in this example may be similar to those for a bicycle and motorbike, since many mopeds can be operated by a mixture of pedalling and an engine power, although the specific calculations may vary due, for example, to mopeds often having a smaller wheel size than motorbikes.
Quadbike and other All Terrain Vehicles (ATVs)—these vehicles can give rise to some of the same considerations as a motorbike, for example fork angle. However, there are other issues to consider, in particular lateral stability, which is not subject to gyroscopic forces in the way that a two-wheel vehicle is. For at least this reason, a quadbike represents a greater tipping risk on rough or uneven ground than a motorbike or bicycle and so in some implementations, tilt measurements could be processed with terrain measurements and adjustments made to try to reduce tipping risk. For example, as well as or instead of making adjustments to alter the centre of gravity, for example in dependence on incline, a wheel base adjustment could be made, for example if the terrain ahead were deemed likely to cause the vehicle to roll.
Skateboard—successful riding of a skateboard is highly dependent on the rider adjusting his or her body position in anticipation of significant inclines or declines and corners. One parameter that affects stability is the wheel spacing, especially in relation to the location of weight-related forces imposed by the user at foot contact points. Thus a combination of measurements on the user's body and terrain knowledge could be used to affect an adjustment of the wheelbase, either by adjusting a length of a component on which the wheels are held fixed relative thereto, or by adjusting the length or width of the deck. A distance of the wheels from the deck can also influence cornering and this could be adjusted by making adjustments to one or more wheel spacers, for example by increasing their wedge angle.
Pram—the manoeuvrability of a pram can depend on some similar parameters as those discussed above with respect to other vehicles and could therefore be made adjustable. Some examples include fork angle (especially on a 3-wheel pram) and wheel spacing. As well as actions such as cornering, the need to negotiate steps could be considered and, for example, centre of gravity adjusted to make this easier for the user pushing the pram.
Wheelchair—similar considerations to those of a pram can apply in this example. However, centre of gravity calculations may be adjusted to allow for different user-occupant limb lengths and masses, especially if the occupant is an adult.

Some alternatives to particular implementations described above have already been mentioned. Regarding the screen 136 and user interfaces 138 and 133, alternatives will occur to the skilled reader. For example, information regarding adjustments which could or are going to be made could be provided to the user as any form of sensory input, including auditory feedback and tactile feedback. User inputs may be received in any form, including, but not limited to, acoustic, speech, or tactile input. Possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like. Some alternatives to the LIDAR terrain scanner described above include image capture devices, whose images could be analyzed to provide similar information as a LIDAR scanner, and sound-reflection-based capture devices.

The functions of the controller 62 described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor such as the microprocessor 124, which can be special or general purpose, coupled to receive data and instructions from, and to transmit data and instructions to storage devices such as sticks and devices on other vehicles. Such computer programs, which can be software, software applications, applications, components, or code, include machine instructions for a programmable processor such as the microprocessor 124, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor and which can receive instructions as a machine-readable signal. Such a machine-readable medium can store instructions transitorily or non-transitorily.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A vehicle comprising:
a frame component having an adjustable physical property; and
a controller configured to receive data relating to the vehicle in use and, based on received data, determine a value of the physical property of the frame component.

2. A vehicle in accordance with claim 1, wherein the controller is further configured to compare a current value of the physical property to the determined value, and if different, determine an adjustment of the frame component to cause it to have substantially the determined value.

3. A vehicle in accordance with claim 2, wherein the controller is configured to generate instructions relating to the determined adjustment, the vehicle further comprising an actuator arranged to adjust the frame component based on the instructions.

4. A vehicle in accordance with any preceding claim, wherein the received data relates to properties of any one of: the vehicle; a terrain on which the vehicle is being used; a terrain ahead of the vehicle; a user of the vehicle; and data from another vehicle which is crossing the terrain generally ahead of the vehicle.

5. A vehicle in accordance with any preceding claim, wherein the data is received in real-time and/or uploaded to the controller prior to use of the vehicle.

6. A vehicle in accordance with any preceding claim, further comprising one or more sensors arranged to gather information during use of the vehicle, use the information to generate data and provide data to the controller in real-time.

7. A vehicle in accordance with claim 6, wherein one or more of the sensors is a terrain sensor, arranged to gather information about a terrain ahead of the vehicle.

8. A vehicle in accordance with claim 7, wherein the terrain sensor is arranged to gather information comprising one or more of: incline or decline; presence of an obstacle; smoothness; hardness; surface topography; surface friction and climate.

9. A vehicle in accordance with any of claims 6 to 8, wherein one or more of the sensors is arranged to measure one or more parameters of the vehicle.

10. A vehicle in accordance with claim 9, wherein the vehicle parameters comprise one or more of: vehicle component lengths; forces sustained by vehicle components; angle between vehicle components; gearing selections; vehicle speed; vehicle acceleration; angle of vehicle relative to the horizontal in a direction of travel; tilt or roll angle of vehicle; angle of vehicle components; distance between vehicle components; forces applied to user interface components; speed of user interface components; tire pressures; and acceleration of user interface components.

11. A vehicle in accordance with any of claims 6 to 10, wherein one or more of the sensors is arranged to measure one or more parameters of a user of the vehicle.

12. A vehicle in accordance with claim 11, wherein the user parameters comprise one or more of: heart rate; temperature; blood pressure; blood sugar levels; muscle fatigue; breath composition; position of the user relative to the vehicle; and angle of user body parts relative to the vehicle or each other.

13. A vehicle in accordance with any preceding claim, wherein the received data comprises one or more of: previously-generated terrain data pertaining to a terrain on which the vehicle is to travel; data from the vehicle or another vehicle captured from a previous travel across the terrain; vehicle component masses; vehicle component inertias; vehicle component compliances; suspension component settings; user mass; user segment inertias; user segment lengths; user gender; user fitness; user strength; user preferences for feel of vehicle; user preference for component stiffness; user preference for suspension hardness; user preference for vehicle dimension; and information input by the user.

14. A vehicle in accordance with any preceding claim, wherein the value of the physical property affects one or more of the following factors: comfort; handling; speed; controllability and efficiency of the user of the vehicle.

15. A vehicle in accordance with claim 14, wherein the controller is configured to determine the value of the physical property to optimize one or more of the factors in accordance with user demand.

16. A vehicle in accordance with any preceding claim, wherein the physical property of the frame component comprises a dimension of the component.

17. A vehicle in accordance with claim 16, wherein the dimension is adjustable by means of one or more of: an extensible portion; an elastic portion; the component having two parts overlapping to an adjustable degree; a mechanical fuse; an unfoldable portion; an additional portion that can be selectively incorporated into the component; the component being moveable relative another component; adjustment of an attachment of the component to another component; selective deployment of one of multiple alternative components; a rack and pinion; a worm gear; an inerter; and a travel limiter on the component.

18. A vehicle in accordance with any preceding claim, wherein the physical property comprises a compliance of the frame component.

19. A vehicle in accordance with claim 18, wherein the compliance is adjustable by means of one or more of: a chargeable portion; and a selectively deployable supporting portion.

20. A vehicle in accordance with any preceding claim, wherein the frame component is one or more of: a supporting structural component; a rear triangle component; a front triangle component; a saddle post; a handlebar; a fork; and a wheel spacer.

21. A method comprising:
receiving data relating to use of a vehicle; and
determining, based on received real-time data, a value of a physical property of a frame component of the vehicle.

22. A method in accordance with claim 1, further comprising comparing a current value of the physical property to the determined value and if different, determining an adjustment of the frame component to cause it to have substantially the determined value.

23. A method in accordance with claim 22, further comprising adjusting the frame component in accordance with the determined adjustment.

24. A method in accordance with any of claims 21 to 23, wherein the received data relates to properties of any one of the vehicle; a terrain on which the vehicle is being used; a terrain ahead of the vehicle; a user of the vehicle; and data from another vehicle which is crossing the terrain generally ahead of the vehicle.

25. A method in accordance with any of claims 21 to 24, further comprising receiving some or all of the data in real-time and/or uploading some or all of the data prior to use of the vehicle.

26. A method in accordance with any of claims 21 to 25, further comprising:
gathering information during use of the vehicle; and
using the information to generate data.

27. A method in accordance with claim 26, wherein gathering information comprises gathering information about a terrain ahead of the vehicle.

28. A method in accordance with claim 27, wherein the information comprises one or more of: incline or decline; presence of an obstacle; smoothness; hardness; surface topography; surface friction and climate.

29. A method in accordance with any of claims 26 to 28, wherein gathering information comprises measuring one or more parameters of the vehicle.

30. A method in accordance with claim 29, wherein the vehicle parameters comprise one or more of: vehicle component lengths; forces sustained by vehicle components; angle between vehicle components; gearing selections; vehicle speed; vehicle acceleration; angle of vehicle relative to the horizontal in a direction of travel; tilt or roll angle of vehicle; angle of vehicle components; distance between vehicle components; forces applied to user interface components; speed of user interface components; tire pressure; and acceleration of user interface components.

31. A method in accordance with any of claims 26 to 30, wherein gathering information comprises measuring one or more parameters of a user of the vehicle.

32. A method in accordance with claim 31, wherein the user parameters comprise one or more of: heart rate; temperature; blood pressure; blood sugar levels; muscle fatigue; breath composition; position of the user relative to the vehicle; and angle of user body parts relative to the vehicle or each other.

33. A method in accordance with any of claims 21 to 32, wherein the received data comprises one or more of: previously-generated terrain data pertaining to a terrain on which the vehicle is to travel; data from the vehicle or another vehicle captured from a previous travel across the terrain; vehicle component masses; vehicle component inertias; vehicle component compliances; suspension component settings; user mass; user segment inertias; user segment lengths; user gender; user fitness; user strength; user preferences for feel of vehicle; user preference for component stiffness; user preference for suspension hardness; user preference for vehicle dimension; and information input by the user.

34. A method in accordance with any of claims 21 to 33, wherein the value of the physical property affects one or more of the following factors: comfort; handling; speed; controllability and efficiency of the user of the vehicle.

35. A method in accordance with claim 34, wherein determining the value of the physical property comprises optimizing one or more of the factors in accordance with user demand.

36. A method in accordance with any of claims 21 to 33, wherein the physical property of the structural component comprises a dimension of the component.

37. A method in accordance with claim 36, wherein the dimension is adjustable by means of one or more of: an extensible portion; an elastic portion; the component having two parts overlapping to an adjustable degree; a mechanical fuse; an unfoldable portion; an additional portion that can be selectively incorporated into the component; the component being moveable relative another component; adjustment of an attachment of the component to another component; selective deployment of one of multiple alternative components; a rack and pinion; a worm gear; an inerter; and a travel limiter on the component.

38. A method in accordance with any of claims 21 to 37, wherein the physical property comprises a compliance of the structural component.

39. A method in accordance with claim 38, wherein the compliance is adjustable by means of one or more of: a chargeable portion; and a selectively deployable supporting portion.

40. A method in accordance with any of claims 21 to 39, wherein the frame component is one or more of: a supporting structural component; a rear triangle component; a front triangle component; a saddle post; a handlebar; a fork; and a wheel spacer.

41. A vehicle or method in accordance with any preceding claim, wherein the vehicle is one of: a bicycle; a motorbike and/or sidecar; a moped; an all terrain vehicle (ATV); a skateboard; a pram; and a wheelchair.

42. A computer program product comprising a computer-readable medium storing instructions which, when executed by at least one programmable processor, cause the at least one programmable processor to perform operations comprising the method of any of claims 21 to 41.

43. A control system for adjusting a structural component of a vehicle, comprising a computer-readable medium in accordance with claim 42 and one or more actuators arranged to adjust the structural component based on the determined value.

44. A vehicle substantially as herein described with reference to the accompanying drawings.

45. A method substantially as herein described with reference to the accompanying drawings.
